SEQUENCE LISTING (62 sequences)

SEQ ID NO: 1 - DNA, 2419 bp, Artificial Sequence
  A nucleotide sequence of one of the chains of the Alb enhancer-Alb promoter target fragment
ggtggttctcctgtcagtttcgagggggtacagcttgggctgcaggtcgactctagatcgaattcctgcagcccgggggatcccggggttgataggaaaggtgatctgtgtgcagaaagactcgctctaatatacttctttaaccaataactgtagatcattaaccatacttacctcgcatttcattggttcctaccccattacaaaatcataccatctttgccaaaaagttgtttgactaaatcccttgcgtatgtttgccatctggagctgttcccctctaaccccacccccacccccatgcacaagactttgtccattcattaaagttatgtaaaacagcaaattttacataagagcttaatctctttgtctcccatttgagcatttcagtgtgggccttggcatggaagcatgcctgcaggtcgatcccaagctggagaacgagttcaagccaagctgcaccactgcttttcacacactcttcactctgcatcagcttagtatttcttaagaaattaaaagatggcaaaacacatctaaactgtattaataaagtgcttctttcatatttaatgtttttccagataaagaaaactatgatgaatgcctgcatgcttatctatgtttcatagatcagcaagtagaatgtataaaatggaagtgtcagtaattctgctcataattattgctgcagattgaattcacccctaagcaaatatacctctgaacatctgctcacagtctgtatgttctccagacacaatccaaaagacttattatctgaaagattaatgtcacaaagccagagctttataatctcttataaaacatagattgtagccaggcagtggtggcacatgcttttaatcctagcacttgcaaggcagaggcaagcagatctctgagttcaagaccaacctggtctacagagcaaggtccaggacagccaaagctagacagaaaaaactgtatctcaaaagaaaatagacaacaaattacattgttacagctaaaattatcttatgttgaaatttctgtagctcaactttggaatattttcattagagggtaatatttgattatgatcacttctaaaactttagaatttattgttttataatctcttggtttcagtacttacctaaaattttccaaccagtcacccagctaaaacttaaaatatttaagtcctagaattccagttagttttgcaagtaactataaatggtattacagtgagaaatggagcatctgatgtctactcacatgtaaactttacacatatcaaatagatgattgtctatggtctttcttcttttttagagtatatagagtatatagagatagattcatccataataagctcaataaacaaatgtttaaaaatgattgttagatattattgggtatataagtacctaattattaaaattgacttttttataacattgagataaattaaaattcatttattaaaataatatatatgaatttgaaggggttttttttgcaaaacaatttcagcaagcaataccatgacaaaagtgtgtattcaaatggaatgggaaacgaatgtcagtaacttatggtccccgtgtactcattcccagacatgcctgattggtagctgtgacagctccagcgtacttaacaccaagactttaaataagctgccaaaaatgtgtaagactgccatttcattagttttaatttttatatctataccctttctacagccacatactaaacgtagacaagttggccttttcctattgctttaaaggcagaggactgtattgatcagtccaaacttctttctgcatgtacatggaaaactggccaaggcaaacacgtccggaatgatggtatttaagaacaaacattccctggtatcagcaagtacagtgccctgctgacagagcaggagacacaaagtaccatctcgtccctatgttaagtagtgtcacctcatgctcaagggatactgagtggatgctgtaacgcaggttattttctaggctgtgaggatacaagaaaatgaaagtaattaaagtagaacattgctctgtgctatgcttgcagaatgtgtagtgtagtctaggaacagagaggggaaggttctaaatcaaaaaaaatcaagctcatgcctaaggatgtgtgggttgccacctctttagctacctatgcgatccaaacaactataaaacttagaatttattttctctggatgaatttgtgcttgtggagcaatgttggtagggggcagggtcagctggaaaagtggaatgagcaagcagaaaactgagagaagcagaagcttaggaagatgggtaatttccaaaagtttcacaaaagatcaaatcaaagaagtaagctccaccttagaaaaaagtggaacgtcatgctaaggaagcta

SEQ ID NO: 2 - DNA, 747 bp, Artificial Sequence
  A nucleotide sequence of one of the chains of the Tet-On 3G target fragment
ttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacat

SEQ ID NO: 3 - DNA, 635 bp, Artificial Sequence
  A nucleotide sequence of one of the chains of the HGH polyA target fragment
ggctgcaggaattcaacaggcatctactgagtggacccaacgcatgagaggacagtgccaagcaagcaactcaaatgtcccaccggttgggccatggcaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccatc

SEQ ID NO: 4 - DNA, 1352 bp, Artificial Sequence
  A nucleotide sequence of one of the chains of the Rosa26 arm1
ttggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttgcagtctggtacttccaagctcattagatgccatcatgctctcactgcctcctcagcttcaagaggaatctggaaaaagcagtcccactggtcaggaaaggaacactagtgcacttatc

SEQ ID NO: 5 - DNA, 1094 bp, Artificial Sequence
  A nucleotide sequence of one of the chains of the Rosa26 arm2
tgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgat

SEQ ID NO: 6 - DNA, 1715 bp, Artificial Sequence
  A nucleotide sequence of one of the chains of the uPA-rabbit polyA target fragment
atgaaagtctggctggcgagcctgttcctctgcgccttggtggtgaaaaactctgaaggtggcagtgtacttggagctcctgatgaatcaaactgtggctgtcagaacggaggtgtatgcgtgtcctacaagtacttctccagaattcgccgatgcagctgcccaaggaaattccagggggagcactgtgagatagatgcatcaaaaacctgctatcatggaaatggtgactcttaccgaggaaaggccaacactgataccaaaggtcggccctgcctggcctggaatgcgcctgctgtccttcagaaaccctacaatgcccacagacctgatgctattagcctaggcctggggaaacacaattactgcaggaaccctgacaaccagaagcgaccctggtgctatgtgcagattggcctaaggcagtttgtccaagaatgcatggtgcatgactgctctcttagcaaaaagccttcttcgtctgtagaccaacaaggcttccagtgtggccagaaggctctaaggccccgctttaagattgttgggggagaattcactgaggtggagaaccagccctggttcgcagccatctaccagaagaacaagggaggaagtcctccctcctttaaatgtggtgggagtctcatcagtccttgctgggtggccagtgccgcacactgcttcattcaactcccaaagaaggaaaactacgttgtctacctgggtcagtcgaaggagagctcctataatcctggagagatgaagtttgaggtggagcagctcatcttgcacgaatactacagggaagacagcctggcctaccataatgatattgccttgctgaagatacgtaccagcacgggccaatgtgcacagccatccaggtccatacagaccatctgcctgcccccaaggtttactgatgctccgtttggttcagactgtgagatcactggctttggaaaagagtctgaaagtgactatctctatccaaagaacctgaaaatgtccgtcgtaaagcttgtttctcatgaacagtgtatgcagccccactactatggctctgaaattaattataaaatgctgtgtgctgcggacccagagtggaaaacagattcctgcaagggcgattctggaggaccgcttatctgtaacatcgaaggccgcccaactctgagtgggattgtgagctggggccgaggatgtgcagagaaaaacaagcccggtgtctacacgagggtctcacacttcctggactggattcaatcccacattggagaagagaaaggtctggccttctgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactact

SEQ ID NO: 7 - protein, 433 aa, Artificial Sequence
  An amino acid sequence of uPA
MKVWLASLFLCALVVKNSEGGSVLGAPDESNCGCQNGGVCVSYKYFSRIRRCSCPRKFQGEHCEIDASKTCYHGNGDSYRGKANTDTKGRPCLAWNAPAVLQKPYNAHRPDAISLGLGKHNYCRNPDNQKRPWCYVQIGLRQFVQECMVHDCSLSKKPSSSVDQQGFQCGQKALRPRFKIVGGEFTEVENQPWFAAIYQKNKGGSPPSFKCGGSLISPCWVASAAHCFIQLPKKENYVVYLGQSKESSYNPGEMKFEVEQLILHEYYREDSLAYHNDIALLKIRTSTGQCAQPSRSIQTICLPPRFTDAPFGSDCEITGFGKESESDYLYPKNLKMSVVKLVSHEQCMQPHYYGSEINYKMLCAADPEWKTDSCKGDSGGPLICNIEGRPTLSGIVSWGRGCAEKNKPGVYTRVSHFLDWIQSHIGEEKGLAF

SEQ ID NO: 8 - DNA, 379 bp, Artificial Sequence
  A nucleotide sequence of the TRE3G fragment
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 9 - DNA, 20 bp, Artificial Sequence
  The target sequence of sgRNA used for the targeting vector
agtcttctgggcaggcttaa

SEQ ID NO: 10 - DNA, 1382 bp, Artificial Sequence
  A nucleotide sequence of one of the chains of the Tet-On 3G-HGH polyA fusion target fragment
ggctgcaggaattcaacaggcatctactgagtggacccaacgcatgagaggacagtgccaagcaagcaactcaaatgtcccaccggttgggccatggcaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccatcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacat

SEQ ID NO: 11 - DNA, 2094 bp, Artificial Sequence
  A nucleotide sequence of one of the chains of the TRE3G-uPA-rabbit polyA fusion fragment
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaatgaaagtctggctggcgagcctgttcctctgcgccttggtggtgaaaaactctgaaggtggcagtgtacttggagctcctgatgaatcaaactgtggctgtcagaacggaggtgtatgcgtgtcctacaagtacttctccagaattcgccgatgcagctgcccaaggaaattccagggggagcactgtgagatagatgcatcaaaaacctgctatcatggaaatggtgactcttaccgaggaaaggccaacactgataccaaaggtcggccctgcctggcctggaatgcgcctgctgtccttcagaaaccctacaatgcccacagacctgatgctattagcctaggcctggggaaacacaattactgcaggaaccctgacaaccagaagcgaccctggtgctatgtgcagattggcctaaggcagtttgtccaagaatgcatggtgcatgactgctctcttagcaaaaagccttcttcgtctgtagaccaacaaggcttccagtgtggccagaaggctctaaggccccgctttaagattgttgggggagaattcactgaggtggagaaccagccctggttcgcagccatctaccagaagaacaagggaggaagtcctccctcctttaaatgtggtgggagtctcatcagtccttgctgggtggccagtgccgcacactgcttcattcaactcccaaagaaggaaaactacgttgtctacctgggtcagtcgaaggagagctcctataatcctggagagatgaagtttgaggtggagcagctcatcttgcacgaatactacagggaagacagcctggcctaccataatgatattgccttgctgaagatacgtaccagcacgggccaatgtgcacagccatccaggtccatacagaccatctgcctgcccccaaggtttactgatgctccgtttggttcagactgtgagatcactggctttggaaaagagtctgaaagtgactatctctatccaaagaacctgaaaatgtccgtcgtaaagcttgtttctcatgaacagtgtatgcagccccactactatggctctgaaattaattataaaatgctgtgtgctgcggacccagagtggaaaacagattcctgcaagggcgattctggaggaccgcttatctgtaacatcgaaggccgcccaactctgagtgggattgtgagctggggccgaggatgtgcagagaaaaacaagcccggtgtctacacgagggtctcacacttcctggactggattcaatcccacattggagaagagaaaggtctggccttctgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactact

SEQ ID NO: 12 - DNA, 1140 bp, Artificial Sequence
  A nucleotide sequence of one of the chains of the H11 arm1
ctcagcagacacccaggataagtgcactagtgttcctttcctgaccagtgggactgctttttccagattcctcttgaagctgaggaggcagtgagagcatgatggcatctaatgagcttggaagtaccagactgccctgatccacagccaggttttgctgaaaagtgaccagtttgtcctcctccagtagagtgggcagctgaaggattataatctactgtcaagacttggaggcccctgcagtcaaagtccaatagaatattatgaaatggagaatggcttattttaatctctatagtggaattaaaatagcatttatggccccagatccataattaatccaatgactggtcaaattagcttgaacctgactgaaaaacctgcaatcagtatagtatctttcagaatgctttacattcaatttataataccagacttcaagttgtaagttaacaattttgagagaaactaatgcagcagaggcaagggaaagacttaaattaatgtgaccattaattagttggagtcaggctggattaacattgtgccagtaaatttcagaaaaattactatatgacttctctgtaaactttgattttgtagagtataatattgattccttgatacttgccacaagctactttcagggttagtccagcttaaactaatgctatcagaactttatgtgcagaagaaattccagaagtcctaaggtaaaattaaaacgtgaaaggaactcatttagactgtcttagttcatggaagaaataaacacagtaccagccatatcgttcacacacgtggcatatgtaacttttaataaaccaaaagaaaaagtcccaaatattcaagtgaaaaaaatccaaacagttgacacaagcctaactgatagttactttatgtacagctgtatgtataagttcaaaaaaagctaccctatttgtatgtacaaaagtttatacacaggtctgtacataagggtctatacattttattttttcagaacccttaggtgtcacctctagaagacaccaacacttcattcacatattttataaaagaaagacttccaggactgacaatcttgtatcccttgtatttgaaccatgtaggttcattcgatttaacagccttctgtca

SEQ ID NO: 13 - DNA, 1057 bp, Artificial Sequence
  A nucleotide sequence of one of the chains of the H11 arm2
tagctcaccttgaaaatggaaacatgtctgacaagagccttgagctgaatatcatccagagtatttgctagagacagggtcttaagtctcattaaattgcttagaagtgtgtttagtcctataaactatgtctcatttgtgtgtccttcacaaagagcgctagtgtcgatcatccattagcctagcctataaaggtgacacaacctggtcagtccctctgtatgtctactatttccccttctgatttctactatcttctcaagaactggttcttcagcttcctttgggaaatgtcactttttaatgatgtgtgttttgcttatgtatatgtctacatactacccatgtgcttggtcactgcagaggccagaagacggtgtcaggtgcactggaactagagttaatgacagatgtgagccatagggtgctttgttcacatctttccagtcccaaatgcgtctaaacttatgtgatacatactagattaccacaaaattgctccaaagacacctttctccctctgagatgaatctctggctggccttgctctaggcaatcctgtgttcaattcaaggactgaatttgattacatcggtaatccagtgccccaggcatttctgctttttctgtaaggttcttatcccctggaagactgtttacgtattcatttttttttgagactaagtttcaggtagaaaaagcttgccttgaacttcactatatagggcttatcttgaactcttgtgggtcttccacctttcttcagttagcttctgtacactgccagacatgaaaatcagatccatttatagatagatggtcatgagccaccatgtgggtgtctaaaattgatctcaggacctctgaaagaccagctagttctcttaactgctgagccatctctctagcgtgtctatacacatttaatatccccttgttccctttctgcttcatcttgctgatcatgattagtgtttgcctttgttacctgttccatcagcttcagcctgaagagtaagtagttctctattggcagtttgacacatcctgcccttac

SEQ ID NO: 14 - DNA, 20 bp, Artificial Sequence
  The target sequence of sgRNA used for the control targeting vector
ctgagccaacagtggtagta

SEQ ID NO: 15 - DNA, 54 bp, Artificial Sequence
  Enhancer-promoter-R
gtgatctgcaactccagtctttggcgcgcctagcttccttagcatgacgttcca

SEQ ID NO: 16 - DNA, 42 bp, Artificial Sequence
  Enhancer-promoter-F
ccagtctagacatggcggctttgccagaggctagtggggttg

SEQ ID NO: 17 - DNA, 35 bp, Artificial Sequence
  TetOn3G-F
aagccgccaccatgtctagactggacaagagcaaa

SEQ ID NO: 18 - DNA, 45 bp, Artificial Sequence
  TetOn3G-R
tcacagggatgccacccatcttacccggggagcatgtcaaggtca

SEQ ID NO: 19 - DNA, 40 bp, Artificial Sequence
  HGH polyA-F
ttgacatgctccccgggtaagatgggtggcatccctgtga

SEQ ID NO: 20 - DNA, 53 bp, Artificial Sequence
  HGH polyA-R-BamHI
cctgcaggacaacgcccacacaggatccggctgcaggaattcaacaggcatct

SEQ ID NO: 21 - DNA, 35 bp, Artificial Sequence; identical to SEQ ID NO: 17
  TetOn3G-F

SEQ ID NO: 22 - DNA, 53 bp, Artificial Sequence; identical to SEQ ID NO: 20
  HGH polyA-R-BamHI

SEQ ID NO: 23 - DNA, 22 bp, Artificial Sequence
  Alb-seq-F1
ggtgcaagcacgtttccgactt

SEQ ID NO: 24 - DNA, 22 bp, Artificial Sequence
  Alb-seq-R1
tctgtgctatgcttgcagaatg

SEQ ID NO: 25 - DNA, 22 bp, Artificial Sequence
  Alb-seq-F6
atcatatgtggcctggagaaac

SEQ ID NO: 26 - DNA, 22 bp, Artificial Sequence
  Alb-seq-R7
ggagcgggagaaatggatatga

SEQ ID NO: 27 - DNA, 48 bp, Artificial Sequence
  uPA-rabbit polyA-R- BamHI
gtgatctgcaactccagtctttggatccagtagtcaggagaggaggaa

SEQ ID NO: 28 - DNA, 70 bp, Artificial Sequence
  uPA-rabbit polyA-F
tcttataccaactttccgtaccacttcctaccctcgtaaagccgccaccatgaaagtctggctggcgagc

SEQ ID NO: 29 - DNA, 61 bp, Artificial Sequence
  TRE3G-F
gaacgtcatgctaaggaagctagagtttactccctatcagtgatagagaacgtatgaagag

SEQ ID NO: 30 - DNA, 71 bp, Artificial Sequence
  TRE3G-R
aaggcgcagaggaacaggctcgccagccagactttcatggtggcggctttacgagggtaggaagtggtacg

SEQ ID NO: 31 - DNA, 61 bp, Artificial Sequence; identical to SEQ ID NO: 29
  TRE3G-F

SEQ ID NO: 32 - DNA, 46 bp, Artificial Sequence
  uPA-rabbit polyA-R- BamHI
gtgatctgcaactccagtctttggatcctagtcaggagaggaggaa

SEQ ID NO: 33 - DNA, 22 bp, Artificial Sequence
  Alb-seq-F1
ggtgcaagcacgtttccgactt

SEQ ID NO: 34 - DNA, 22 bp, Artificial Sequence
  Alb-seq-R8
ccccttgagcatctgacttctg

SEQ ID NO: 35 - DNA, 22 bp, Artificial Sequence
  Alb-seq-F1
ggtgcaagcacgtttccgactt

SEQ ID NO: 36 - DNA, 22 bp, Artificial Sequence
  Alb-seq-F2
cctagactacactacacattct

SEQ ID NO: 37 - DNA, 22 bp, Artificial Sequence
  Alb-seq-R3
agattgaattcacccctaagca

SEQ ID NO: 38 - DNA, 22 bp, Artificial Sequence
  Alb-seq-F4
tgccaccactgcctggctacaa

SEQ ID NO: 39 - DNA, 22 bp, Artificial Sequence
  Alb-seq-F5
gaggtaagtatggttaatgatc

SEQ ID NO: 40 - DNA, 22 bp, Artificial Sequence
  Alb-seq-F6
atcatatgtggcctggagaaac

SEQ ID NO: 41 - DNA, 22 bp, Artificial Sequence
  Alb-seq-R7
ggagcgggagaaatggatatga

SEQ ID NO: 42 - DNA, 22 bp, Artificial Sequence
  Alb-seq-F8
tccaacacactattgcaatgaa

SEQ ID NO: 43 - DNA, 22 bp, Artificial Sequence
  Alb-seq-F9
tgacccaggtagacaacgtagt

SEQ ID NO: 44 - DNA, 22 bp, Artificial Sequence
  Alb-seq-F10
aggctcgccagccagactttca

SEQ ID NO: 45 - DNA, 38 bp, Artificial Sequence
  H11-F-BglII
gaagatctggcgcgccgtaagggcaggatgtgtcaaac

SEQ ID NO: 46 - DNA, 38 bp, Artificial Sequence
  H11-R-BglII
gaagatctggcgcgccctcagcagacacccaggataag

SEQ ID NO: 47 - DNA, 22 bp, Artificial Sequence
  Alb-H11-seq-F1
tgcaaggcgattaagttgggta

SEQ ID NO: 48 - DNA, 22 bp, Artificial Sequence
  Alb-seq-R8
ccccttgagcatctgacttctg

SEQ ID NO: 49 - DNA, 22 bp, Artificial Sequence
  Alb-H11-seq-F2
ttgctgggattacaggcgtgaa

SEQ ID NO: 50 - DNA, 22 bp, Artificial Sequence
  Alb-H11-seq-R2
gcgggcagtgagcgcaacgcaa

SEQ ID NO: 51 - DNA, 23 bp, Artificial Sequence
  H11-tF3
gggcagtctggtacttccaagct

SEQ ID NO: 52 - DNA, 22 bp, Artificial Sequence
  Alb-tet3G-TRE3G-TR1
gtgtgggttgccacctctttag

SEQ ID NO: 53 - DNA, 23 bp, Artificial Sequence
  H11-tF3
gggcagtctggtacttccaagct

SEQ ID NO: 54 - DNA, 23 bp, Artificial Sequence
  H11-tR3
atatccccttgttccctttctgc

SEQ ID NO: 55 - DNA, 25 bp, Artificial Sequence
  H11-tF2
atgcccaccaaagtcatcagtgtag

SEQ ID NO: 56 - DNA, 22 bp, Artificial Sequence
  Alb-tet3G-TRE3G-5TR1
gtgtgggttgccacctctttag

SEQ ID NO: 57 - DNA, 25 bp, Artificial Sequence
  Alb-tet3G-TRE3G -3tF2
gcgatctgacggttcactaaacgag

SEQ ID NO: 58 - DNA, 23 bp, Artificial Sequence
  H11-tR2
tcacagaaaccatatggcgctcc

SEQ ID NO: 59 - DNA, 23 bp, Artificial Sequence
  upA-tF1
tggagctcctgatgaatcaaact

SEQ ID NO: 60 - DNA, 20 bp, Artificial Sequence
  upA-tR1
atagcaccagggtcgcttct

SEQ ID NO: 61 - DNA, 20 bp, Artificial Sequence
  Tet on3G-F1
acaggcatcatacccactcc

SEQ ID NO: 62 - DNA, 18 bp, Artificial Sequence
  Tet on3G-R2
gcgtcagcaggcagcata

(19) United States
(12) Patent Application Publication
(10) Pub. No.: US 2022/0142130 A1
(43) Pub. Date: May 12, 2022

(54) Targeting Vector, Nucleic Acid Composition, and Method for Constructing Liver-injured Mouse Model

(71) Applicant: GEMPHARMATECH CO., LTD., Jiangsu (CN)

(72) Inventors: Cunxiang JU, Jiangsu (CN); Dongjing JIA, Jiangsu (CN); Jing ZHAO, Jiangsu (CN)

(73) Assignee: GEMPHARMATECH CO., LTD., Jiangsu (CN)

(21) Appl. No.: 17/611,882

(22) PCT Filed: Oct. 20, 2020
(86) PCT No.: PCT/CN2020/122228

§ 371 Date: Nov. 16, 2021

(30) Foreign Application Priority Data
Aug. 3, 2020 (CN) 202010764997.3

(51) Int. Cl. A01K 67/027; C12N 15/85; C12N 15/90

(52) U.S. Cl. CPC A01K 67/0276; C12N 15/85; C12N 15/907; C12N 2830/008; A01K 2227/105; A01K 2267/0393; C12N 2830/003; A01K 2217/075

(57) ABSTRACT

Provided are a targeting vector, a nucleic acid composition, and a method for constructing a liver-injured mouse model. The targeting vector includes a first expression cassette and a second expression cassette located downstream of the first expression cassette, the first expression cassette has the following elements connected in series in sequence: a liver-specific promoter, a tetracycline transcription activation regulating factor, and a first polyA; and the second expression cassette has the following elements connected in series in sequence: a second polyA, a mouse prourokinase activator encoding gene, and a tetracycline-inducible promoter. The liver-injured mouse model constructed with this targeting vector has the phenotype of spontaneously generating the liver injury and aggravating the liver injury by induction, which provides liver-injured mouse models for studies of liver diseases.

### CROSS-REFERENCE TO RELATED APPLICATION

This application is a national stage application of PCT/CN2020/122228 filed on Oct. 20, 2020, which claims priority to Chinese patent application with the filing No. 202010764997.3 filed on Aug. 3, 2020 with the Chinese Patent Office, entitled “Targeting Vector, Nucleic Acid Composition, and Method for Constructing Liver-injured Mouse Model”, the contents of which are incorporated herein by reference in its entirety.

### TECHNICAL FIELD

The present disclosure relates to the technical field of gene engineering and genetic modification of genes, in particular, to a targeting vector, a nucleic acid composition, and a method for constructing a liver-injured mouse model.

### BACKGROUND ART

Liver diseases are one of the most serious diseases threatening human health, especially hepatitis B (HBV), hepatitis C (HCV), cirrhosis, liver cancer, non-alcoholic fatty liver disease (NAFLD), alcoholic liver disease (ALD), and drug-induced liver injury (DILI), among which viral hepatitis is the most widespread. The viral hepatitis has hepatotropism, with strong species specificity and tissue specificity. For example, natural hosts of hepatitis B virus and hepatitis C virus are merely limited to human beings and a few non-human primates (chimpanzees), and they only infect the liver tissues of the host. However, the study of viral hepatitis using chimpanzees is restricted both ethically and economically. Due to the complexity of human liver diseases and the lack of suitable animal models, medical research and transformation of human liver diseases are limited. As being chimerized with the human hepatocytes, the liver-humanized mouse model can simulate the process of human infecting with hepatitis virus, thus leading to more in-depth research of liver diseases.

In drug research and development, whether a drug is safe or not is judged through pharmacokinetics/pharmacodynamics (PK/PD) of preclinical animal experiments and human body experiments, which has guiding significance for the success of clinical tests for the drug. However, many drug metabolizing enzymes have species specificity, and the liver metabolic function of rats or mice are different from that of human beings, then the drug PK/PD test using rats or mice cannot truly reflect the metabolism of the drug in the human body, therefore, it is possible to cause extremely high safety risk by the drug on the human body. In order to elucidate the drug metabolic pathway in the human body, human hepatocytes are needed for tests. Compared with in vitro human hepatocytes metabolism experiments, the liver-humanized mice can more truly reflect the metabolic pathway of the drug in the human body.

The basis of constructing the liver-humanized mice is that there must be mice with liver injury as receptors, to receive implantation of the human hepatocytes, and the human hepatocytes proliferate and develop in the mouse body into functional human-mouse chimeric livers. Currently, common liver-injured models include uPA-SCID, Fah^{-/-}Rag2^{-/-}II2rg^{-/-}(FRG), TK-NOG, Alb-uPA, etc. Although the human liver chimerism rates of these models have been reported to be able to reach 90%, these models still have some drawbacks that limit their application. For example, uPA-SCID, Alb-uPA, and FRG models cause frequent death of offspring mice in perinatal period due to hepatotoxicity, resulting in quite high mortality of newborn mice, then large-scale breeding is difficult. The male mice of TK-NOG are sterile, so that the model is difficult to breed and passage, and the use of the model is limited; after uPA-SCID mice are reconstructed, the replacement index of human hepatocytes (h-heps) is reduced due to deletion of uPA gene of homologous recombination, then kidney diseases are likely to occur. Deng Hongkui realized the Tet on-uPA recombination system in mouse bodies utilizing mice carrying different vector fragments combined with adenovirus carrying uPA gene for transfection, or multiplying two models carrying different vectors (but the breeding process was quite tedious). In theory, the controllable uPA expression timing improves the mouse death. However, as repeated adenovirus infection is required for maintaining the uPA expression, the host mice are likely to generate resistance, then the subsequent adenovirus infection rate is affected, the uPA expression is reduced, and then the hepatocyte reconstruction efficiency is reduced. In addition, natural immunity can be activated after the model is injected with the adenovirus, which affects the observation of immune response induced by hepatitis virus, and is not suitable for infection research of hepatitis virus.

Due to the above limitations, there is a great need in the field for a suitable liver-injured mouse model for construction of humanized liver, and for subsequent liver disease research and drug screening using the liver-humanized mouse model.

### SUMMARY

The present disclosure provides a targeting vector for constructing a liver-injured mouse model, wherein the targeting vector contains a target sequence and a 5′ end homologous arm sequence and a 3′ end homologous arm sequence for mediating insertion of the target sequence into a target site in a mouse genome; and the target sequence includes a first expression cassette and a second expression cassette located downstream of the first expression cassette;

the first expression cassette has the following elements connected sequentially in series: a liver-specific promoter, a tetracycline transcription activation regulating factor, and a first polyA; and the second expression cassette has the following elements connected sequentially in series: a second polyA, a mouse prourokinase activator encoding gene, and a tetracycline-inducible promoter.

In the above, the liver-specific promoter drives the expression of the tetracycline transcription activation regulating factor in a direction from upstream to downstream, and the tetracycline-inducible promoter drives the expression of the mouse prourokinase activator encoding gene in a direction from downstream to upstream.

In one or more embodiments, the first polyA is constituted by connecting 1-3 identical or different polyA. In one or more embodiments, the number of A (adenylic acid) in each polyA is 50-200.

In one or more embodiments, the second polyA is constituted by connecting 1-3 identical or different polyA. In one or more embodiments, the number of A (adenylic acid) in each polyA is 50-200.

In one or more embodiments, the first expression cassette further has an enhancer sequence; and the enhancer sequence is located upstream of the liver-specific promoter.

In one or more embodiments, the liver-specific promoter is any one selected from the group consisting of albumin promoter, apolipoprotein E promoter, phosphoenolpyruvate carboxykinase promoter, α-I-antitrypsin promoter, thyroxin binding globulin promoter, α-fetoprotein promoter, alcohol dehydrogenase promoter, IGF-II promoter, factor VIII promoter, HBV core protein promoter, HBV pre-s2 protein promoter, thyroxine-binding globulin promoter, HCR-ApOCII hybrid promoter, HCR-hAAT hybrid promoter, AAT promoter combined with enhancer element of mouse albumin gene, low-density lipoprotein promoter, pyruvate kinase promoter, lecithin-cholesterol acyltransferase promoter, apolipoprotein H promoter, transferrin promoter, transthyretin promoter, α-fibrinogen and β-fibrinogen promoter, α-I-antichymotrypsin promoter, α-2-HS glycoprotein promoter, haptoglobin promoter, ceruloplasmin promoter, plasminogen promoter, complement protein promoter, promoter of complement C3 activator, hemopexin promoter, and α-I-acid glycoprotein promoter.

In one or more embodiments, the liver-specific promoter is an albumin promoter.

In one or more embodiments, the enhancer sequence is an albumin enhancer.

In one or more embodiments, the tetracycline transcription activation regulating factor is any one selected from the group consisting of tTA, rtTA, and Tet-On 3G.

In one or more embodiments, the tetracycline transcription activation regulating factor is Tet-On 3G.

In one or more embodiments, the first polyA is selected from the group consisting of HGH polyA, SV40 polyA, BGH polyA, rbGlob polyA, SV40 late polyA, and rbGlob polyA.

In one or more embodiments, in the second expression cassette, a Kozak sequence is further inserted between the mouse prourokinase activator encoding gene and the tetracycline-inducible promoter.

In one or more embodiments, the tetracycline-inducible promoter is any one selected from the group consisting of TRE3G and TetO6.

In one or more embodiments, the tetracycline-inducible promoter is TRE3G.

In one or more embodiments, an amino acid sequence of the mouse prourokinase activator encoded by the mouse prourokinase activator encoding gene is represented by SEQ ID NO. 7.

In one or more embodiments, a nucleotide sequence of the mouse prourokinase activator encoding gene is represented by sites 1-1302 in SEQ ID NO. 6 or a complementary sequence thereof.

In one or more embodiments, the second polyA is selected from the group consisting of rabbit polyA, SV40 polyA, hGH polyA, BGH polyA, rbGlob polyA, SV40 late polyA, and rbGlob polyA.

In one or more embodiments, the target site is Rosa26 site.

In one or more embodiments, the 5′ end homologous arm sequence is represented by SEQ ID NO. 4 or a complementary sequence thereof; and the 3′ end homologous arm sequence is represented by SEQ ID NO. 5 or a complementary sequence thereof.

The present disclosure provides a nucleic acid composition for constructing a liver-injured mouse model, including the targeting vector described herein and a CRISPR/Cas9 composition for occurring double-strand breakage of a mouse genome sequence at the target site.

In one or more embodiments, the CRISPR/Cas9 composition includes: Cas9 protein and sgRNA.

In one or more embodiments, the target sequence of the sgRNA is represented by SEQ ID NO. 9.

The present disclosure provides a recombinant cell, wherein it contains the targeting vector described herein.

The present disclosure provides a kit for constructing a liver-injured mouse model, wherein the kit includes the targeting vector described herein, or a nucleic acid composition described herein.

The present disclosure provides a method for constructing a liver-injured mouse model, wherein the target sequence is inserted into the target site on the genome of a target mouse using the targeting vector described herein or the nucleic acid composition described herein.

In one or more embodiments, the target mouse is a mouse having immunodeficiency.

In one or more embodiments, the method includes: injecting the nucleic acid composition into fertilized eggs from mice having immunodeficiency, then transplanting the fertilized eggs into pseudo-pregnant female mouse bodies, and screening out, from offspring of the pseudo-pregnant female mice, positive mice with the genome inserted with the target sequence, to thus obtain the liver-injured mouse models.

The present disclosure provides a breeding method of a liver-injured mouse model, including: the liver-injured mouse model obtained by the construction method described herein mating with a wild-type mouse having immunodeficiency.

The present disclosure provides use of the liver-injured mouse model obtained by the construction method described herein or the liver-injured mouse model obtained by the breeding method described herein in screening drugs for the treatment of liver diseases, and the use is for the purpose of non-disease diagnosis or treatment.

In one or more embodiments, the use includes: implanting human hepatocytes into the liver-injured mouse model for liver humanization, and then using the liver-injured mouse model having undergone the liver humanization for screening drugs.

The present disclosure provides a method of screening a drug for treatment of liver diseases, including:

providing the liver-injured mouse model obtained by the construction method described herein or the liver-injured mouse model obtained by the breeding method described herein,

implanting human hepatocytes into the liver-injured mouse model for liver humanization, and

administering a candidate drug to the liver-injured mouse model having undergone the liver humanization.

The present disclosure provides the use described herein or the method described herein, wherein the liver diseases are selected from the group consisting of viral hepatitis, liver fibrosis, cirrhosis, fatty liver, pharmaceutical liver injury, and liver cancer.

The present disclosure provides use of the targeting vector described herein for constructing the liver-injured mouse model.

### BRIEF DESCRIPTION OF DRAWINGS

In order to more clearly illustrate technical solutions of examples of the present disclosure, accompanying drawings which need to be used in the examples will be introduced below briefly, and it should be understood that the accompanying drawings below merely show some examples of the present disclosure, and therefore should not be considered as limitation on the scope, and a person ordinarily skilled in the art still could obtain other relevant accompanying drawings according to these accompanying drawings, without using any creative efforts.

FIG. 1 is a structural schematic diagram of a target sequence of a targeting vector in Example 1.

FIG. 2 is a graph of gel electrophoresis results for PCR identification of partial bacterial solution containing intermediate vectors in Example 2.

FIG. 3 is a graph of gel electrophoresis results for enzyme digestion identification of partial intermediate vectors in Example 2 (DL is DL2000 standard (marker), band sizes are 2000, 1000, 750, 500, 200, 100 bp, respectively; and T14 is EcoT14I standard (marker), band sizes are 19329, 7743, 6223, 4254, 3472, 2690, 1882, 1489 bp, respectively).

FIG. 4 is a graph of gel electrophoresis results for PCR identification of a bacterial solution containing a targeting vector in Example 2.

FIG. 5 is a graph of gel electrophoresis results for enzyme digestion identification of the targeting vector in Example 2.

FIG. 6 is a structural schematic diagram of a control targeting vector in Example 3.

FIG. 7 is a graph of gel electrophoresis results for PCR identification of a bacterial solution containing the control targeting vector in Example 3.

FIG. 8 is a graph of gel electrophoresis results for enzyme digestion identification of the control targeting vector in Example 3.

FIG. 9 is a graph of gel electrophoresis results of partial F1 generation H11-alb-Tet On3G-uPA mice having undergone PCR identification (A: identification results detected for Rosa26-alb-Tet-On3G-uPA mice positive and wt; B: identification results detected for 3′ end and 5′ end of targeting sequences of Rosa26-alb-Tet-On3G-uPA mice positive).

FIG. 10 is a graph of gel electrophoresis results of partial F1 generation Rosa26-alb-Tet On3G-uPA mice having undergone PCR identification (A: graph of gel electrophoresis results of PCR identification of mouse No. 2; B: graph of gel electrophoresis results of PCR identification of mouse No. 15).

FIG. 11 shows detection results of liver injury law of H11-alb-Tet On3G-uPA mice induced by doxycycline (Dox) (A: H11-alb-Tet On3G-uPA heterozygote mice are administered with Dox drinking water to induce liver injury; B: H11-alb-Tet On3G-uPA homozygous mice are administered with Dox drinking water or intragastric administration (PO) to induce liver injury).

FIG. 12 shows detection result of glutamic-pyruvic transaminase (ALT) activity of spontaneous liver injury of Rosa26-alb-Tet On3G-uPA heterozygote mice.

FIG. 13 shows HE staining results of Rosa26-alb-Tet On3G-uPA mice at 4 weeks of age, showing severe liver injury.

FIG. 14 shows detection results of 3-4-week-old Rosa26-alb-Tet On3G-uPA mice administered with Dox to induce and aggravate liver injury degree (A: ALT changes in serum of 3-4-week-old mice after being administered with Dox for induction; B: liver HE staining of 3-4-week-old mice after being administered with Dox for 7 days for induction).

FIG. 15 shows detection results of 6-8-week-old Rosa26-alb-Tet On3G-uPA mice administered with Dox to induce and aggravate liver injury degree (A: ALT changes in serum of 6-8-week-old mice after being administered with Dox for induction; B: liver HE staining of 6-8-week-old mice after being administered with Dox 7 days for induction).

FIG. 16 shows liver morphology 10 weeks after transplantation of green fluorescent hepatocytes to Rosa26-alb-Tet On3G-uPA mice of different weeks of age (green fluorescent hepatocytes are uniformly distributed on the liver surface).

FIG. 17 shows observation results of green fluorescent cells 10 weeks after transplantation of hepatocytes to Rosa26-alb-Tet On3G-uPA mice of different weeks of age.

FIG. 18 is a pRosa26-Cas plasmid vector profile.

FIG. 19 is a PMD18T-H11-CAG-FLPo plasmid vector profile.

FIG. 20 shows detection result of ALT activity of spontaneous liver injury of Rosa26-alb-Tet On3G-uPA homozygote mice.

FIG. 21 shows positional relationships of respective elements (two expression cassettes are in tail-to-tail ligation in A, two expression cassettes are in tail-to-head ligation in B, two expression cassettes are in head-to-head ligation in C, and GFP plasmid expresses green fluorescence in D).

FIG. 22 shows that after electrotransformation of GFP plasmid into HepG2 cells, the cells can express green fluorescence.

FIG. 23 shows the influence on cells after electrotransformation of plasmids having two cassettes in different ligation relationships into HepG2 cells.

FIG. 24 shows that green fluorescence can be expressed in hepatocytes after a mouse is injected with GFP plasmid via tail vein.

FIG. 25 shows ALT levels of the liver-injured mice induced by plasmids having two expression cassettes in different ligation relationships.

### DETAILED DESCRIPTION OF EMBODIMENTS

In order to make the objectives, technical solutions, and advantages of the examples of the present disclosure clearer, the technical solutions in the examples of the present disclosure will be described below clearly and completely. If no specific conditions are specified in the examples, they are carried out under normal conditions or conditions recommended by manufacturers. If manufacturers of reagents or apparatuses used are not specified, they are conventional products commercially available.

Objectives of one or more embodiments of the present disclosure include, for example, providing a targeting vector, a nucleic acid composition, and a method for constructing a liver-injured mouse model. The liver-injured mouse model constructed using the targeting vector provided in the present disclosure can generate liver injury without induction of an inducer, it can spontaneously generate the liver injury, and the degree of liver injury can be enhanced with use of the inducer. In addition, the degree of spontaneous liver injury of the liver-injured mouse model not only can satisfy the transplantation requirements of exogenous hepatocytes, but also the reconstruction rate is high after hepatocytes transplantation. Besides, the liver-injured mouse model can breed offspring liver-injured mice by crossbreeding, and the mortality of offspring mice is low, facilitating large-scale breeding. The present disclosure provides a reliable liver-injured mouse model for studies of liver diseases.

The present disclosure provides a targeting vector for constructing a liver-injured mouse model, wherein the targeting vector contains a target sequence and a 5′ end homologous arm sequence and a 3′ end homologous arm sequence for mediating insertion of the target sequence into a target site in a mouse genome; and the target sequence includes a first expression cassette and a second expression cassette located downstream of the first expression cassette;

the first expression cassette has the following elements connected sequentially in series: a liver-specific promoter, a tetracycline transcription activation regulating factor, and a first polyA; the second expression cassette has the following elements connected sequentially in series: a second polyA, a mouse prourokinase activator encoding gene, and a tetracycline-inducible promoter;

in the above, the liver-specific promoter drives the expression of the tetracycline transcription activation regulating factor in a direction from upstream to downstream, and the tetracycline-inducible promoter drives the expression of the mouse prourokinase activator encoding gene in a direction from downstream to upstream.

In one or more embodiments, the first expression cassette and the second expression cassette of the target sequence are linked through the ligation between respective polyA, i.e., constituting tail-to-tail ligation.

In one or more embodiments, the first expression cassette and the second expression cassette of the target sequence are linked through the ligation between respective polyA, and a transcription direction of the first expression cassette is opposite to that of the second expression cassette.

In one or more embodiments, the first expression cassette and the second expression cassette of the target sequence are linked through the ligation between respective promoters, i.e. constituting head-to-head ligation.

In one or more embodiments, the first expression cassette and the second expression cassette of the target sequence are linked through the ligation between respective promoters, and a transcription direction of the first expression cassette is opposite to that of the second expression cassette.

In one or more embodiments, the first expression cassette and the second expression cassette of the target sequence are linked through the ligation between respective promoters, and a transcription direction of the first expression cassette is the same as that of the second expression cassette, i.e. constituting tail-to-head ligation.

In one or more embodiments, the target sequence includes a liver-specific promoter, a tetracycline transcription activation regulating factor, a first polyA, a second polyA, a mouse prourokinase activator encoding gene, and a tetracycline-inducible promoter arranged in sequence.

As used herein, “polyA” is also called as a polyadenine nucleotide, and generally refers to a chain consisting of a plurality of adenine nucleotides. In general, the number of adenine nucleotides in polyA is 50-200.

The above target sequence can be inserted into the target site in the mouse genome using the targeting vector provided in the present disclosure. The ligation relationship between the first expression cassette and the second expression cassette in the above target sequence may be construed as tail-to-tail ligation. The inventors have surprisingly found that mice having the genome inserted with the target sequence having the first expression cassette and the second expression cassette with the above ligation manner and elements have the following unexpected phenotypes or characteristics, and can be used as a relatively ideal liver-injured mouse models.

(1) The liver injury may be generated without induction of an inducer, the liver injury can spontaneously generated, and the degree of liver injury can be enhanced with use of the inducer, therefore, users may make selective induction or no induction according to transplantation requirement, then the transplantation procedure is simplified. This spontaneous liver injury phenotype breaks the conventional knowledge of characteristics for the Tet on system to regulate protein expression, and this phenotype greatly goes beyond the expected range of the inventors.

(2) The degree of spontaneously generated liver injury of the liver-injured mouse model is sufficient to meet the transplantation requirements of exogenous hepatocytes; and if the induction is further performed, the degree of liver injury is more severe, then the reconstruction rate after transplantation of exogenous hepatocytes is higher.

(3) The liver-injured mouse model may breed more offspring liver-injured mice by crossbreeding, and the mortality of the offspring mice is much lower than that of the same type of mouse model with spontaneous liver injury, which facilitates large-scale breeding of liver-injured mouse models, and the phenotype also greatly goes beyond the expected range of the inventors.

It should be noted that, in some other examples, positions of the first expression cassette and the second expression cassette may be exchanged, i.e. the second expression cassette is upstream, the first expression cassette is downstream, the sequence of positions of the elements are correspondingly adjusted, the second expression cassette is driven downstream, i.e. in a direction towards the first expression cassette, and the first expression cassette is driven upstream, i.e. in a direction towards the second expression cassette. This configuration can also achieve similar effects. That is, no matter what direction is arranged, it is feasible as long as the polyAs of the two expression cassettes are adjacent, and the two expression cassettes are driven in a direction from two ends to the middle to express.

In an optional embodiment, the first expression cassette further has an enhancer sequence; and the enhancer sequence is located upstream of the liver-specific promoter.

In an optional embodiment, the liver-specific promoter includes, but is not limited to, any one selected from the group consisting of albumin promoter, apolipoprotein E promoter, phosphoenolpyruvate carboxykinase promoter, α-I-antitrypsin promoter, thyroxin binding globulin promoter, α-fetoprotein promoter, alcohol dehydrogenase promoter, IGF-II promoter, factor VIII promoter, HBV core protein promoter, HBV pre-s2 protein promoter, thyroxine-binding globulin promoter, HCR-ApOCII hybrid promoter, HCR-hAAT hybrid promoter, AAT promoter combined with enhancer element of mouse albumin gene, low-density lipoprotein promoter, pyruvate kinase promoter, lecithin-cholesterol acyltransferase promoter, apolipoprotein H promoter, transferrin promoter, transthyretin promoter, α-fibrinogen and β-fibrinogen promoter, α-I-antichymotrypsin promoter, α-2-HS glycoprotein promoter, haptoglobin promoter, ceruloplasmin promoter, plasminogen promoter, complement protein promoter, promoter of complement C3 activator, hemopexin promoter, and α-I-acid glycoprotein promoter.

In an optional embodiment, the liver-specific promoter is an albumin promoter.

In an optional embodiment, the enhancer sequence includes, but is not limited to, an albumin enhancer.

In an optional embodiment, the tetracycline transcription activation regulating factor is any one selected from the group consisting of tTA, rtTA, and Tet-On 3G.

In an optional embodiment, the tetracycline transcription activation regulating factor is Tet-On 3G.

In an optional embodiment, the first polyA includes, but is not limited to, HGH polyA, SV40 polyA, BGH polyA, rbGlob polyA, SV40 late polyA, and rbGlob polyA.

In an optional embodiment, the tetracycline-inducible promoter is any one selected from the group consisting of TRE3G and TetO6.

In an optional embodiment, the tetracycline-inducible promoter is TRE3Gp.

In an optional embodiment, an amino acid sequence of the mouse prourokinase activator encoded by the mouse prourokinase activator encoding gene is represented by SEQ ID NO. 7.

In an optional embodiment, a nucleotide sequence of the mouse prourokinase activator encoding gene is represented by sites 1-1302 in SEQ ID NO. 6 or a complementary sequence thereof.

In an optional embodiment, the second polyA includes, but is not limited to, rabbit polyA, SV40 polyA, hGH polyA, BGH polyA, rbGlob polyA, SV40 late polyA, and rbGlob polyA.

In an optional embodiment, in the second expression cassette, a Kozak sequence is further inserted between the mouse prourokinase activator encoding gene and the tetracycline-inducible promoter.

In an optional embodiment, the target site is Rosa26 site.

Inserting the above target sequence at the Rosa26 site can avoid interference effects of adjacent sequences on the genome, and will not disrupt any endogenous genes, thereby ensuring normal growth and development and normal function of mice; in addition, compared with other common insertion sites (e.g., H11), inserting the above target sequence at the Rosa26 site also enables the liver-injured mouse model to exhibit a unique phenotype, i.e., the degree of liver injury spontaneously generated can satisfy the implantation of exogenous hepatocytes. In addition, the liver-injured mouse model has more sensitive response to inducer (e.g., Dox), and after induction, the degree of liver injury is aggravated. Inserting the target sequence at other sites does not have similar technical effects, and the effect is unexpected to the inventors of the present disclosure.

In an optional embodiment, the 5′ end homologous arm sequence is represented by SEQ ID NO. 4 or a complementary sequence thereof; and the 3′ end homologous arm sequence is represented by SEQ ID NO. 5 or a complementary sequence thereof.

In an optional embodiment, a skeleton of the targeting vector can be selected according to actual needs, and no matter what kind of skeleton vector is selected to carry the above target sequence, it belongs to the scope of protection of the present disclosure.

The present disclosure provides a recombinant cell containing the above targeting vector.

In an optional embodiment, the recombinant cell includes, but is not limited to, E. coli. A person skilled in the art could choose a suitable host cell to transform the above targeting vector so as to facilitate storage or amplification of the above targeting vector, and no matter which host cell is selected, they all belong to the scope of protection of the present disclosure.

The present disclosure provides a nucleic acid composition for constructing a liver-injured mouse model, including the targeting vector according to any one of the above and a CRISPR/Cas9 composition for occurring double-strand breakage of a mouse genome sequence at the target site.

In an optional embodiment, the CRISPR/Cas9 composition includes: Cas9 protein and sgRNA.

In an optional embodiment, the target sequence of the sgRNA is represented by SEQ ID NO. 9.

As used herein, “CRISPR/Cas9 composition” refers to a gene editing system based on clustered regularly interspaced short palindromic repeats (CRISPR) and nuclease. Generally, the CRISPR/Cas9 composition includes sgRNA (small guide RNA) and Cas9 protein, then specific genetic modification can be realized.

The present disclosure provides a kit for constructing a liver-injured mouse model, wherein the kit includes the targeting vector according to any one of the above, or a nucleic acid composition according to any one of the above.

The present disclosure provides a method for constructing a liver-injured mouse model, wherein the target sequence is inserted into the target site on the genome of a target mouse using the targeting vector according to any one of the above or the nucleic acid composition according to any one of the above.

The present disclosure provides use of the targeting vector described herein for constructing the liver-injured mouse model. In one or more embodiments, the use involves inserting the target sequence at the target site on the genome of a target mouse.

The present disclosure provides use of the nucleic acid composition described herein for constructing a liver-injured mouse model. In one or more embodiments, the use involves inserting the target sequence at the target site on the genome of a target mouse.

The present disclosure provides the targeting vector described herein, which is used for constructing a liver-injured mouse model. In one or more embodiments, the use involves inserting the target sequence at the target site on the genome of a target mouse.

The present disclosure provides the nucleic acid composition described herein, which is used for constructing a liver-injured mouse model. In one or more embodiments, the use involves inserting the target sequence at the target site on the genome of a target mouse.

In one or more embodiments, the above use involves mating the liver-injured mouse model obtained by the construction method described herein with a wild-type mouse having immunodeficiency.

The liver-injured mouse model constructed using the construction method provided in the present disclosure can generate liver injury without induction of an inducer, it can spontaneously generate the liver injury, and the degree of liver injury can be enhanced with use of the inducer. In addition, the degree of spontaneous liver injury of the liver-injured mouse model not only can satisfy the transplantation requirements of exogenous hepatocytes, but also the reconstruction rate is high after transplantation. Besides, the liver-injured mouse model can breed offspring liver-injured mice by crossbreeding, and the mortality of offspring mice is low, facilitating large-scale breeding.

In the method for constructing a liver-injured mouse model of the present disclosure, an inducer is further used to treat a target mouse. In one or more embodiments, the inducer is Dox.

In an optional embodiment, the target mouse is a mouse having immunodeficiency (NCG mouse).

In an optional embodiment, the method includes: injecting the nucleic acid composition into fertilized eggs from mice having immunodeficiency, then transplanting the fertilized eggs into pseudo-pregnant female mouse bodies, and screening out, from the offspring of the pseudo-pregnant female mice, positive mice with the genome inserted with the target sequence, to thus obtain the liver-injured mouse model.

In an optional embodiment, the fertilized eggs are developed for 0.5 days.

In an optional embodiment, the pseudo-pregnant female mice are pseudo-pregnant female mice 0.5 days after successful mating.

The present disclosure provides a breeding method of a liver-injured mouse model, including: the liver-injured mouse model obtained by the construction method described above mating with a wild-type mouse having immunodeficiency.

The liver-injured mouse model obtained by the above construction method can reduce the mortality of offspring mice by mating with wild-type mice having immunodeficiency, and offspring liver-injured mice with the same phenotype as the parental liver-injured mice may be obtained on a large scale. In this way, a person skilled in the art could obtain an ideal liver-injured mouse models in large batches by means of a simpler hybridization breeding method, without the need of repeating the above construction method, thus greatly improving the breeding efficiency, and effectively satisfying the requirement for the number of liver-injured mouse models in the art.

The present disclosure provides use of the liver-injured mouse model obtained by the construction method or breeding method as described above in screening drugs for the treatment of liver diseases. In an optional embodiment, the use is for the purpose of non-disease diagnosis or treatment. In another optional embodiment, the use is for the purpose of disease diagnosis or treatment.

The present disclosure provides use of the liver-injured mouse model obtained by the construction method or breeding method as described above in screening drugs for the treatment of liver diseases.

In an optional embodiment, the use includes: implanting human hepatocytes into the liver-injured mouse model for liver humanization, and then using the liver-injured mouse model having undergone the liver humanization for screening drugs.

In an optional embodiment, the liver diseases include, but are not limited to, viral hepatitis (e.g. Hepatitis B and hepatitis C), liver fibrosis, cirrhosis, fatty liver diseases (e.g. alcoholic and non-alcoholic fatty liver diseases), pharmaceutical liver injury, and liver cancer.

The liver-injured mouse model obtained by the targeting vector and the construction method of the present disclosure, after having undergone the implantation of exogenous hepatocytes, for example, human hepatocytes, i.e., liver humanization, can be used to screen drugs for the treatment of liver diseases, for example, evaluating whether candidate drugs are effective or safe for human hepatocytes, and it can reflect the metabolic condition of the candidate drugs in the human body more realistically, and screen out more reliable drugs.

The features and performances of the present disclosure are further described below in detail in combination with examples.

### EXAMPLES

### Example 1

A targeting vector for constructing a liver-injured mouse model provided in the present example included: a target sequence, and a 5′ end homologous arm sequence (Rosa26 arm1) and a 3′ end homologous arm sequence (Rosa26 arm2) for mediating insertion of the target sequence into a target site (Rosa26) in a mouse genome (see FIG. 1 for positional relationship of various elements).

In the above, the target sequence included a first expression cassette and a second expression cassette located downstream of the first expression cassette in sequence in a direction from upstream to downstream;

the first expression cassette included the following elements connected sequentially in series: albumin enhancer (Alb enhancer), albumin promoter (Alb promoter), tetracycline transcription activation regulating factor (Tet-On 3G), and a first polyA (HGH polyA); as for the direction in FIG. 1, the expression was driven from left (upstream) to right (downstream).

The second expression cassette included the following elements connected sequentially in series: a second polyA (rabbit polyA, indicated by pA in FIG. 1), mouse prourokinase activator encoding gene (uPA), Kozak sequence, and tetracycline-inducible promoter (TRE3G); as for the direction in FIG. 1, the expression was driven from right (downstream) to left (upstream).

Rosa26 arm1 was located upstream of the target sequence, and Rosa26 arm2 was located downstream of the target sequence.

The albumin promoter (Alb promoter) drove the expression of the tetracycline transcription activation regulating factor (Tet-On 3G) in a direction from upstream to downstream (see broken line arrow on the left of FIG. 1), and the tetracycline-inducible promoter (TRE3G) drove the expression of the mouse prourokinase activator encoding gene (uPA) in a direction from downstream to upstream (see broken line arrow on the right of FIG. 1).

Through combined use of the targeting vector provided in the present example and the CRISPR/Cas9 composition, the target sequence could be inserted at sites corresponding to the 5′ end homologous arm sequence and the 3′ end homologous arm sequence in the mouse genome (see FIG. 1).

### Example 2

The present example provides a method for constructing a targeting vector of Example 1, including the following steps:

1.1 Preparing Alb enhancer-Alb promoter fragment

Alb enhancer-Alb promoter target fragment was amplified using primers in Table 1 and recovered for later use. Conditions of PCR amplification were set according to common knowledge in the art.

TABLE 1
List of Primers for Amplifying Alb Enhancer-Alb
Promoter

| Name of primer | Primer sequence (5′-3′) |
|---|---|
| enhancer- | GTGATCTGCAACTCCAGTCTTTGGCGCGCCTAGC |
| promoter-R | TTCCTTAGCATGACGTTCCA |
| enhancer- | CCAGTCTAGACATGGTGGCGGCTTTGCCAGAGGC |
| promoter-F | TAGTGGGGTTG |
| Product size | 2403 bp |
| Template | Alb-uPA-teton-final (provided by |
| | GemPharmatech, Co., Ltd., Jiangsu) |

A nucleotide sequence (SEQ ID NO. 1) of one of the chains of the Alb enhancer-Alb promoter target fragment was as follows (5′-3′):

ggtggttctcctgtcagtttcgagggggtacagcttgggctgcaggtcga
ctctagatcgaattcctgcagcccgggggatcccggggttgataggaaag
gtgatctgtgtgcagaaagactcgctctaatatacttctttaaccaataa
ctgtagatcattaaccatacttacctcgcatttcattggttcctacccca
ttacaaaatcataccatctttgccaaaaagttgtttgactaaatcccttg
cgtatgtttgccatctggagctgttcccctctaaccccacccccaccccc
atgcacaagactttgtccattcattaaagttatgtaaaacagcaaatttt
acataagagcttaatctctttgtctcccatttgaccatttcactctccgc
cttcccatccaaccatgcctgcaggtcgatcccaagctggagaacgagtt
caagccaagctgcaccactgcttttcacacactcttcactctgcatcagc
ttagtatttcttaagaaattaaaagatggcaaaacacatctaaactgtat
taataaagtgcttctttcatatttaatgtttttccagataaagaaaacta
tgatgaatgcctgcatgcttatctatgtttcatagatcagcaagtagaat
gtataaaatggaagtgtcagtaattctgctcataattattgctgcagatt
gaattcacccctaagcaaatatacctctgaacatctgctcacagtctgta
tgttctccagacacaatccaaaagacttattatctgaaagattaatgtca
caaagccagagctttataatctcttataaaacatagattgtagccaggca
gtggtggcacatgcttttaatcctagcacttgcaaggcagaggcaagcag
atctctgagttcaagaccaacctggtctacagagcaaggtccaggacagc
caaagctagacagaaaaaactgtatctcaaaagaaaatagacaacaaatt
acattgttacagctaaaattatcttatgttgaaatttctgtagctcaact
ttggaatattttcattagagggtaatatttgattatgatcacttctaaaa
ctttagaatttattgttttataatctcttggtttcagtacttacctaaaa
ttttccaaccagtcacccagctaaaacttaaaatatttaagtcctagaat
tccagttagttttgcaagtaactataaatggtattacagtgagaaatgga
gcatctgatgtctactcacatgtaaactttacacatatcaaatagatgat
tgtctatggtctttcttcttttttagagtatatagagtatatagagatag
attcatccataataagctcaataaacaaatgtttaaaaatgattgttaga
tattattgggtatataagtacctaattattaaaattgacttttttataac
attgagataaattaaaattcatttattaaaataatatatatgaatttgaa
ggggttttttttgcaaaacaatttcagcaagcaataccatgacaaaagtg
tgtattcaaatggaatgggaaacgaatgtcagtaacttatggtccccgtg
tactcattcccagacatgcctgattggtagctgtgacagctccagcgtac
ttaacaccaagactttaaataagctgccaaaaatgtgtaagactgccatt
tcattagttttaatttttatatctataccctttctacagccacatactaa
acgtagacaagttggccttttcctattgctttaaaggcagaggactgtat
tgatcagtccaaacttctttctgcatgtacatggaaaactggccaaggca
aacacgtccggaatgatggtatttaagaacaaacattccctggtatcagc
aagtacagtgccctgctgacagagcaggagacacaaagtaccatctcgtc
cctatgttaagtagtgtcacctcatgctcaagggatactgagtggatgct
gtaacgcaggttattttctaggctgtgaggatacaagaaaatgaaagtaa
ttaaagtagaacattgctctgtgctatgcttgcagaatgtgtagtgtagt
ctaggaacagagaggggaaggttctaaatcaaaaaaaatcaagctcatgc
ctaaggatgtgtgggttgccacctctttagctacctatgcgatccaaaca
actataaaacttagaatttattttctctggatgaatttgtgcttgtggag
caatgttggtagggggcagggtcagctggaaaagtggaatgagcaagcag
aaaactgagagaagcagaagcttaggaagatgggtaatttccaaaagttt
cacaaaagatcaaatcaaagaagtaagctccaccttagaaaaaagtggaa
cgtcatgctaaggaagcta.

The underlined part was the Alb promoter, and the non-underlined part was the Alb enhancer.

1.2 Preparation of Tet-On 3G-HGH polyA fusion fragment

1.2.1 Preparation of Tet-On 3G fragment. Tet-On 3G target fragment was amplified using the primers in Table 2 with pCMV-Tet3G as template and recovered for later use.

TABLE 2
List of Primers for Amplifying Tet-On 3G
Fragment

| Name of primer | Primer sequence (5′-3′) |
|---|---|
| TetOn3G-F | AAGCCGCCACCATGTCTAGACTGGACAAGAGCAA |
| | A |
| TetOn3G-R | TCACAGGGATGCCACCCATCTTACCCGGGGAGCA |
| | TGTCAAGGTCA |
| Product size | 802 bp |
| Template | pCMV-Tet3G (provided by |
| | GemPharmatech, Co., Ltd., Jiangsu) |

A nucleotide sequence (SEQ ID NO. 2) of one of the chains of the Tet-On 3G target fragment was as follows (5′-3′), and a complementary chain thereof was the encoding sequence:

ttacccggggagcatgtcaaggtcaaaatcgtcaagagcgtcagcaggca
gcatatcaaggtcaaagtcgtcaagggcatcggctgggagcatgtctaag
tcaaaatcgtcaagggcgtcggtcggcccgccgctttcgcactttagctg
tttctccaggccacatatgattagttccaggccgaaaaggaaggcaggtt
cggctccctgccggtcgaacagctcaattgcttgtttcagaagtgggggc
atagaatcggtggtaggtgtctctctttcctcttttgctacttgatgctc
ctgttcctccaatacgcagcccagtgtaaagtggcccacggcggacagag
cgtacagtgcgttctccagggagaagccttgctgacacaggaacgcgagc
tgattttccagggtttcgtactgtttctctgttgggcgggtgccgagatg
cactttagccccgtcgcgatgtgagaggagagcacagcggtatgacttgg
cgttgttccgcagaaagtcttgccatgactcgccttccagggggcaggag
tgggtatgatgcctgtccagcatctcgattggcagggcatcgagcagggc
ccgcttgttcttcacgtgccagtacagggtaggctgctcaactcccagct
tttgagcgagtttccttgtcgtcaggccttcgataccgactccattgagt
aattccagagcagagtttatgactttgctcttgtccagtctagacat.

1.2.2 Preparation of HGH polyA fragment. HGH polyA target fragment was amplified using the primers in Table 3 with pRosa26-Cas-CAG HGH as template and recovered for later use.

TABLE 3
List of Primers for Amplifying HGH polyA
Fragment

| Name of primer | Primer sequence (5′-3′) | Note |
|---|---|---|
| HGH polyA-F | TTGACATGCTCCCCGGGTAAGATGGGT | |
| | GGCATCCCTGTGA | |
| HGH polyA-R- | CCTGCAGGACAACGCCCACACAGGATC | BamHI |
| BamHI | CGGCTGCAGGAATTCAACAGGCATCT | site |
| Product size | 677 bp | |
| Template | pRosa26-Cas-CAG HGH | |
| | (provided by GemPharmatech, | |
| | Co., Ltd., Jiangsu) | |

A nucleotide sequence (SEQ ID NO. 3) of one of the chains of the HGH polyA target fragment was as follows (5′-3′):

ggctgcaggaattcaacaggcatctactgagtggacccaacgcatgagag
gacagtgccaagcaagcaactcaaatgtcccaccggttgggccatggcag
gtagcctatgctgtgtctggacgtcctcctgctggtatagttattttaaa
atcagaaggacagggaagggagcagtggttcacgcctgtaatcccagcaa
tttgggaggccaaggtgggtagatcacctgagattaggagttggagacca
gcctggccaatatggtgaaaccccgtctctaccaaaaaaacaaaaattag
ctgagcctggtcatgcatgcctggaatcccaacaactcgggaggctgagg
caggagaatcgcttgaacccaggaggcggagattgcagtgagccaagatt
gtgccactgcactccagcttggttcccaatagaccccgcaggccctacag
gttgtcttcccaacttgccccttgctccataccacccccctccaccccat
aatattatagaaggacacctagtcagacaaaatgatgcaacttaatttta
ttaggacaaggctggtgggcactggagtggcaacttccagggccaggaga
ggcactggggaggggtcacagggatgccacccatc.

1.2.3 Preparation of Tet-On 3G-HGH polyA fragment. The target fragment Tet-On 3G-HGH polyA was amplified in a manner of fusion PCR using primers in Table 4 with Tet-On 3G and HGH polyA as templates and recovered.

TABLE 4
List of Primers for Fusion PCR for Tet-On 3G-HGH
polyA Fragment

| Name of primer | Primer sequence (5′-3′) | Note |
|---|---|---|
| TetOn3G-F | AAGCCGCCACCATGTCTAGACTGGACA | |
| | AGAGCAAA | |
| HGH polyA- | CCTGCAGGACAACGCCCACACAGGATC | BamHI |
| R-BamHI | CGGCTGCAGGAATTCAACAGGCATCT | site |
| Product size | 1439 bp | |
| Template | Tet On3G and HGH polyA | |

A nucleotide sequence (SEQ ID NO. 10) of one of the chains of the Tet-On 3G-HGH polyA fusion target fragment was as follows (5′-3′):

ggctgcaggaattcaacaggcatctactgagtggacccaacgcatgagag
gacagtgccaagcaagcaactcaaatgtcccaccggttgggccatggcag
gtagcctatgctgtgtctggacgtcctcctgctggtatagttattttaaa
atcagaaggacagggaagggagcagtggttcacgcctgtaatcccagcaa
tttgggaggccaaggtgggtagatcacctgagattaggagttggagacca
gcctggccaatatggtgaaaccccgtctctaccaaaaaaacaaaaattag
ctgagcctggtcatgcatgcctggaatcccaacaactcgggaggctgagg
caggagaatcgcttgaacccaggaggcggagattgcagtgagccaagatt
gtgccactgcactccagcttggttcccaatagaccccgcaggccctacag
gttgtcttcccaacttgccccttgctccataccacccccctccaccccat
aatattatagaaggacacctagtcagacaaaatgatgcaacttaatttta
ttaggacaaggctggtgggcactggagtggcaacttccagggccaggaga
ggcactggggaggggtcacagggatgccacccatcttacccggggagcat
gtcaaggtcaaaatcgtcaagagcgtcagcaggcagcatatcaaggtcaa
agtcgtcaagggcatcggctgggagcatgtctaagtcaaaatcgtcaagg
gcgtcggtcggcccgccgctttcgcactttagctgtttctccaggccaca
tatgattagttccaggccgaaaaggaaggcaggttcggctccctgccggt
cgaacagctcaattgcttgtttcagaagtgggggcatagaatcggtggta
ggtgtctctctttcctcttttgctacttgatgctcctgttcctccaatac
gcagcccagtgtaaagtggcccacggcggacagagcgtacagtgcgttct
ccagggagaagccttgctgacacaggaacgcgagctgattttccagggtt
tcgtactgtttctctgttgggcgggtgccgagatgcactttagccccgtc
gcgatgtgagaggagagcacagcggtatgacttggcgttgttccgcagaa
agtcttgccatgactcgccttccagggggcaggagtgggtatgatgcctg
tccagcatctcgattggcagggcatcgagcagggcccgcttgttcttcac
gtgccagtacagggtaggctgctcaactcccagcttttgagcgagtttcc
ttgtcgtcaggccttcgataccgactccattgagtaattccagagcagag
tttatgactttgctcttgtccagtctagacat.

The underlined part was HGH polyA, and the non-underlined part was Tet-On 3G.

1.3 Preparation of intermediate vector (containing Rosa26 arm1-Alb enhancer-Alb promoter-Tet-On 3G-HGH polyA-Rosa26 arm2 fragment).

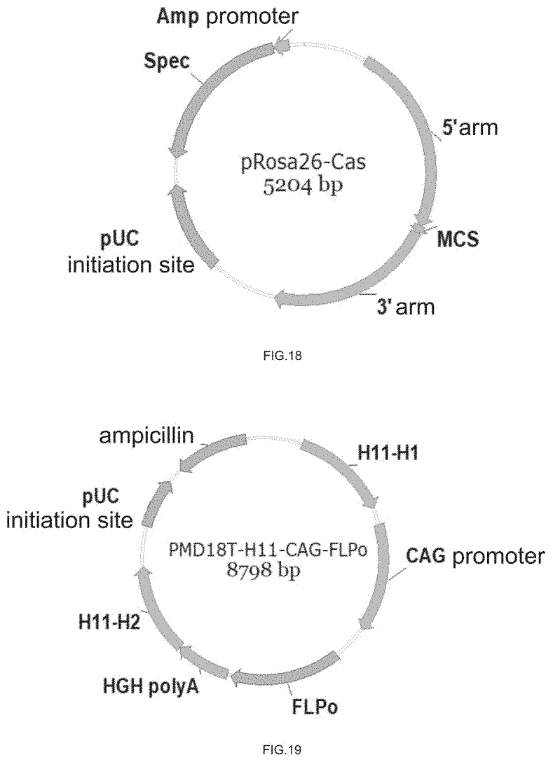

1.3.1 The pRosa26-Cas plasmid was digested with Ascl enzyme (provided by GemPharmatech, Co. Ltd., Jiangsu, wherein the vector itself contains Rosa26 arm1 and Rosa26 arm2, see FIG. 18 for the map), and was recovered as a ligation vector, and the plasmid contained Rosa26 arm1 and Rosa26 arm2 sequences.

A nucleotide sequence (SEQ ID NO. 4) of one of the chains of the Rosa26 arm1 was as follows:

ttggccggtgcgccgccaatcagcggaggctgccggggccgcctaaagaa
gaggctgtgctttggggctccggctcctcagagagcctcggctaggtagg
ggatcgggactctggcgggagggcggcttggtgcgtttgcggggatgggc
ggccgcggcaggccctccgagcgtggtggagccgttctgtgagacagccg
ggtacgagtcgtgacgctggaaggggcaagcgggtggtgggcaggaatgc
ggtccgccctgcagcaaccggagggggagggagaagggagcggaaaagtc
tccaccggacgcggccatggctcgggggggggggggcagcggaggagcgc
ttccggccgacgtctcgtcgctgattggcttcttttcctcccgccgtgtg
tgaaaacacaaatggcgtgttttggttggcgtaaggcgcctgtcagttaa
cggcagccggagtgcgcagccgccggcagcctcgctctgcccactgggtg
gggcgggaggtaggtggggtgaggcgagctggacgtgcgggcgcggtcgg
cctctggcggggcgggggaggggagggagggtcagcgaaagtagctcgcg
cgcgagcggccgcccaccctccccttcctctgggggagtcgttttacccg
ccgccggccgggcctcgtcgtctgattggctctcggggcccagaaaactg
gcccttgccattggctcgtgttcgtgcaagttgagtccatccgccggcca
gcgggggcggcgaggaggcgctcccaggttccggccctcccctcggcccc
gcgccgcagagtctggccgcgcgcccctgcgcaacgtggcaggaagcgcg
cgctgggggcggggacgggcagtagggctgagcggctgcggggcgggtgc
aagcacgtttccgacttgagttgcctcaagaggggcgtgctgagccagac
ctccatcgcgcactccggggagtggagggaaggagcgagggctcagttgg
gctgttttggaggcaggaagcacttgctctcccaaagtcgctctgagttg
ttatcagtaagggagctgcagtggagtaggcggggagaaggccgcaccct
tctccggaggggggaggggagtgttgcaatacctttctgggagttctctg
ctgcctcctggcttctgaggaccgccctgggcctgggagaatcccttccc
cctcttccctcgtgatctgcaactccagtctttgcagtctggtacttcca
agctcattagatgccatcatgctctcactgcctcctcagcttcaagagga
atctggaaaaagcagtcccactggtcaggaaaggaacactagtgcactta
tc.

A nucleotide sequence (SEQ ID NO. 5) of one of the chains of the Rosa26 arm2 was as follows:

tgtgtgggcgttgtcctgcaggggaattgaacaggtgtaaaattggaggg
acaagacttcccacagattttcggttttgtcgggaagttttttaataggg
gcaaataaggaaaatgggaggataggtagtcatctggggttttatgcagc
aaaactacaggttattattgcttgtgatccgcctcggagtattttccatc
gaggtagattaaagacatgctcacccgagttttatactctcctgcttgag
atccttactacagtatgaaattacagtgtcgcgagttagactatgtaagc
agaattttaatcatttttaaagagcccagtacttcatatccatttctccc
gctccttctgcagccttatcaaaaggtattttagaacactcattttagcc
ccattttcatttattatactggcttatccaacccctagacagagcattgg
cattttccctttcctgatcttagaagtctgatgactcatgaaaccagaca
gattagttacatacaccacaaatcgaggctgtagctggggcctcaacact
gcagttcttttataactccttagtacactttttgttgatcctttgccttg
atccttaattttcagtgtctatcacctctcccgtcaggtggtgttccaca
tttgggcctattctcagtccagggagttttacaacaatagatgtattgag
aatccaacctaaagcttaactttccactcccatgaatgcctctctccttt
ttctccatttataaactgagctattaaccattaatggtttccaggtggat
gtctcctcccccaatattacctgatgtatcttacatattgccaggctgat
attttaagacattaaaaggtatatttcattattgagccacatggtattga
ttactgcttactaaaattttgtcattgtacacatctgtaaaaggtggttc
cttttggaatgcaaagttcaggtgtttgttgtctttcctgacctaaggtc
ttgtgagcttgtattttttctatttaagcagtgctttctcttggactggc
ttgactcatggcattctacacgttattgctggtctaaatgtgat.

1.3.2 SLIC ligation conversion method was adopted, ligation 2×HIFI Mix, linearized vector pRosa26-Cas, and the Alb enhancer-Alb promoter fragment, and Tet-On 3G-HGH polyA fragment prepared in the preceding steps were added to a sterile tube, which was supplemented with sterile water to 20 μL; the resultant was reacted at 50° C. for 30 min to obtain an intermediate vector containing the Rosa26 arm1-Alb enhancer-Alb promoter-Tet-On 3G-HGH polyA-Rosa26 arm2 fragment, after the reaction was ended, Top 10 chemical competence transformation was performed, LB solid agar medium with Spec resistance was coated after Top 10 chemical competence transformation was finished, followed by inverted culturing at 37° C. overnight.

1.3.3 Identification of Intermediate Vector.

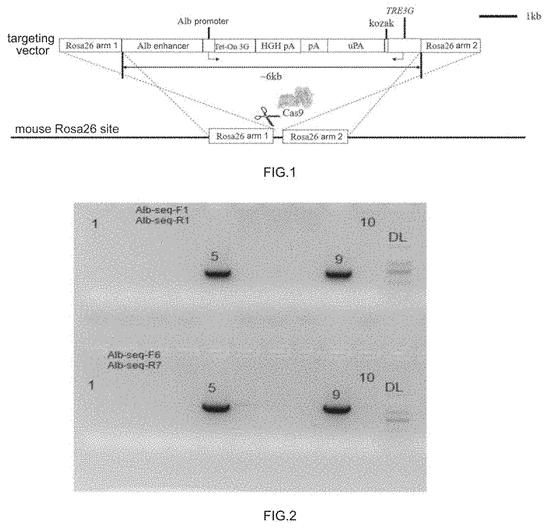

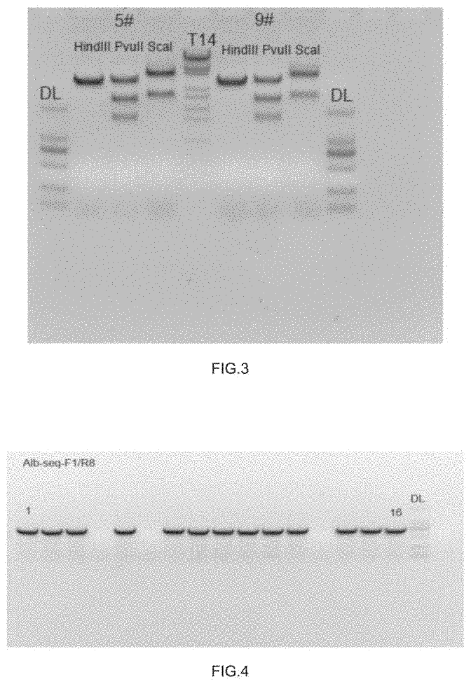

The monoclonal was picked from a plate into test tubes containing 4 mL LB with Spec resistance and cultured. The bacterial solution was subjected to PCR identification using the primers in Table 5 (see FIG. 2 for results), positive clones obtained from the PCR identification were then subjected to enzyme digestion (see Table 6 for expected enzyme digestion bands) identification to confirm (see FIG. 3 for results), and the vectors identified to be correct were intermediate vectors (indicated by Alb-pro). PCR and enzyme digestion identification results show that: 5# and 9# are correct intermediate vectors containing the Rosa26 arm1-Alb enhancer-Alb promoter-Tet-On 3G-HGH polyA-Rosa26 arm2 fragment.

TABLE 5
Primers for PCR Verification for Alb-pro Bacterial
Solution

| Name of primer | Primer sequence (5′-3′) | Note |
|---|---|---|
| Alb-seq-F1 | GGTGCAAGCACGTTTCCGACTT | |
| Alb-seq-R1 | TCTGTGCTATGCTTGCAGAATG | |
| Product size | 698 bp | |
| Alb-seq-F6 | ATCATATGTGGCCTGGAGAAAC | |
| Alb-seq-R7 | GGAGCGGGAGAAATGGATATGA | |
| Product size | 1167 bp | |

TABLE 6
Expected Band Sizes of Enzyme Digestion
Identification for Intermediate Vectors

| Name of restriction enzyme | Band size |
|---|---|
| HindIII | 4546, 4363 |
| PvuII | 4904, 2514, 1491 |
| ScaI | 6112, 2797 |

1.4 Preparation of uPA-TRE3G fusion fragment

1.4.1 Preparation of uPA-rabbit polyA fragment. The uPA-rabbit polyA fragment was amplified using the primers in Table 7 with Alb-uPA-teton-final as template and recovered for later use.

TABLE 7
List of Primers for Amplifying uPA-rabbit polyA
Fragment

| Name of primer | Primer sequence (5′-3′) | Note |
|---|---|---|
| uPA-rabbit | GTGATCTGCAACTCCAGTCTTTGGATCC | BamHI |
| polyA-R-BamHI | AGTAGTCAGGAGAGGAGGAA | site |
| uPA-rabbit | TCTTATACCAACTTTCCGTACCACTTCC | |
| polyA-F | TACCCTCGTAAAGCCGCCACCATGAAAG | |
| | TCTGGCTGGCGAGC | |
| Product size | 1785 bp | |
| Template | Alb-uPA-teton-final | |

In the above, the bold underlined part of the uPA-rabbit polyA-F primer is Kozak sequence.

A nucleotide sequence (SEQ ID NO. 6) of one of the chains of the uPA-rabbit polyA target fragment is as follows:

atgaaagtctgctgcgagcctgttcctctgcgccttgtgtgaaaaactct
gaaggtgcagtgtacttggagctcctgatgaatcaaactgtgctgtcaga
acggaggtgtatgcgtgtcctacaagtacttctccagaattcgccgatgc
agctgcccaaggaaattccagggggagcactgtgagatagatgcatcaaa
aacctgctatcatggaaatggtgactcttaccgaggaaaggccaacactg
ataccaaaggtamccctgcctggcctggaatgcgcctgctgtccttcaga
aaccctacaatgcccacagacctgatgctattagcctaggcctggggaaa
cacaattactgcaggaaccctgacaaccagaagcgaccctgtgctatgtg
cagattgcctaaggcagtttgtccaagaatgcatggtgcatgactgctct
cttagcaaaaagccttcttcgtctgtagaccaacaaggcttccagtgtgg
ccagaaggctctaaggccccgctttaagattgttgggggagaattcactg
aggtggagaaccagccctggttcgcagccatctaccagaagaacaaggga
ggaagtcctccctcctttaaatgtggtgggagtctcatcagtccttgctg
gtgccagtgccgcacactgcttcattcaactcccaaagaaggaaaactac
gttgtctacctgggtcagtcgaaggagagctcctataatcctggagagat
gaagtttgaggtggagcagctcatcttgcacgaatactacagggaagaca
gcctgcctaccataatgatattgccttgctgaagatacgtaccagcacgg
gccaatgtgcacagccatccaggtccatacagaccatctgcctgccccca
aggtttactgatgctccgtttgttcagactgtgagatcactggctttgga
aaagagtctgaaagtgactatctctatccaaagaacctgaaaatgtccgt
cgtaaagcttgtttctcatgaacagtgtatgcagccccactactatggct
ctgaaattaattataaaatgctgtgtgctgcggacccagagtggaaaaca
gattcctgcaagggcgattctggaggaccgcttatctgtaacatcgaagg
ccgcccaactctgagtgggattgtgagctggggccgaggatgtgcagaga
aaaacaagcccggtgtctacacgagggtctcacacttcctggactggatt
caatcccacattggagaagagaaaggtctgccttctgagatctttttccc
tctgccaaaaattatggggacatcatgaagccccttgagcatctgacttc
tggctaataaaggaaatttattttcattgcaatagtgtgttggaattttt
tgtgtctctcactcggaaggacatatgggagggcaaatcatttaaaacat
cagaatgagtatttggtttagagtttggcaacatatgcccatatgctggc
tgccatgaacaaaggttggctataaagaggtcatcagtatatgaaacagc
cccctgctgtccattccttattccatagaaaagccttgacttgaggttag
attttttttatattttgttttgtgttatttttttctttaacatccctaaa
attttccttacatgttttactagccagatttttcctcctctcctgactac
t.

The underlined part was an encoding sequence of uPA; and the non-underlined part was a rabbit polyA sequence. An amino acid sequence of uPA was represented by SEQ ID NO. 7 as follows:

mkvwlaslflcalwknseggsvlgapdesncgcqnggvcvsykyfsrirr
cscprkfqgehceidasktcyhgngdsyrgkantdtkgrpclawnapavl
qkpynahrpdaislglgkhnycrnpdnqkrpwcyvqiglrqfvqecmvhd
cslskkpsssvdqqgfqcgqkalrprfkivggeftevenqpwfaaiyqkn
kggsppsfkcggslispcwvasaahcfiqlpkkenyvvylgqskessynp
gemkfeveglilheyyredslayhndiallkirtstgqcaqpsrsiqtic
lpprftdapfgsdceitgfgkesesdylypknlkmswklvsheqcmqphy
ygseinykmLcaadpewktdsckgdsggplicniegrptlsgivswgrgc
aeknkpgvytrvshfldwiqshigeekglaf.

1.4.2 Preparation of TRE3G fragment. TRE3G fragment was amplified using the primers in Table 8 with pTRE3G as template and recovered for later use.

TABLE 8
List of Primers for Amplifying TRE3G Fragment

| Name of primer | Primer sequence (5′-3′) |
|---|---|
| TRE3G-F | GAACGTCATGCTAAGGAAGCTAGAGTT |
| | TACTCCCTATCAGTGATAGAGAACGTA |
| | TGAAGAG |
| TRE3G-R | AAGGCGCAGAGGAACAGGCTCGCCAGC |
| | CAGACTTTCATGGTGGCGGCTTTACGA |
| | GGGTAGGAAGTGGTACG |
| Product size | 448 bp |
| Template | pTRE3G |

A nucleotide sequence (SEQ ID NO. 8) of the TRE3G fragment is as follows:

gagtttactccctatcagtgatagagaacgtatgaagagtttactcccta
tcagtgatagagaacgtatgcagactttactccctatcagtgatagagaa
cgtataaggagtttactccctatcagtgatagagaacgtatgaccagttt
actccctatcagtgatagagaacgtatctacagtttactccctatcagtg
atagagaacgtatatccagtttactccctatcagtgatagagaacgtata
agctttaggcgtgtacggtgggcgcctataaaagcagagctcgtttagtg
aaccgtcagatcgcctggagcaattccacaacacttttgtcttataccaa
ctttccgtaccacttcctaccctcgtaaa.

1.4.3 Preparation of TRE3G-uPA-rabbit polyA fusion fragment. The fragments uPA-rabbit polyA and TRE3G were fused through fusion PCR using the primers in Table 9, and target bands were recovered for later use.

TABLE 9
List of Primers for Amplifying uPA-TRE3GP
Fusion Fragment

| Name of | | |
|---|---|---|
| primer | Primer sequence (5′-3′) | Note |
| TRE3G-F | GAACGTCATGCTAAGGAAGCTAGAGTT | |
| | TACTCCCTATCAGTGATAGAGAACGTA | |
| | TGAAGAG | |
| uPA-rabbit | GTGATCTGCAACTCCAGTCTTTGGATC | BamHI |
| polyA-R- | CAGTAGTCAGGAGAGGAGGAA | site |
| BamHI | | |
| Product | 2147 bp | |
| size | | |
| Template | uPA rabbit polyA and TRE3G | |

A nucleotide sequence (SEQ ID NO. 11) of one of the chains of the TRE3G-uPA-rabbit polyA fusion fragment was as follows:

gagtttactccctatcagtgatagagaacgtatgaagagtttactcccta
tcagtgatagagaacgtatgcagactttactccctatcagtgatagagaa
cgtataaggagtttactccctatcagtgatagagaacgtatgaccagttt
actccctatcagtgatagagaacgtatctacagtttactccctatcagtg
atagagaacgtatatccagtttactccctatcagtgatagagaacgtata
agctttaggcgtgtacggtgggcgcctataaaagcagagctcgtttagtg
aaccgtcagatcgcctggagcaattccacaacacttttgtcttataccaa
ctttccgtaccacttcctaccctcgtaaaatgaaagtctggctggcgagc
ctgttcctctgcgccttggtggtgaaaaactctgaaggtggcagtgtact
tggagctcctgatgaatcaaactgtggctgtcagaacggaggtgtatgcg
tgtcctacaagtacttctccagaattcgccgatgcagctgcccaaggaaa
ttccagggggagcactgtgagatagatgcatcaaaaacctgctatcatgg
aaatggtgactcttaccgaggaaaggccaacactgataccaaaggtcggc
cctgcctggcctggaatgcgcctgctgtccttcagaaaccctacaatgcc
cacagacctgatgctattagcctaggcctggggaaacacaattactgcag
gaaccctgacaaccagaagcgaccctggtgctatgtgcagattggcctaa
ggcagtttgtccaagaatgcatggtgcatgactgctctcttagcaaaaag
ccttcttcgtctgtagaccaacaaggcttccagtgtggccagaaggctct
aaggccccgctttaagattgttgggggagaattcactgaggtggagaacc
agccctggttcgcagccatctaccagaagaacaagggaggaagtcctccc
tcctttaaatgtggtgggagtctcatcagtccttgctgggtggccagtgc
cgcacactgcttcattcaactcccaaagaaggaaaactacgttgtctacc
tgggtcagtcgaaggagagctcctataatcctggagagatgaagtttgag
gtggagcagctcatcttgcacgaatactacagggaagacagcctggccta
ccataatgatattgccttgctgaagatacgtaccagcacgggccaatgtg
cacagccatccaggtccatacagaccatctgcctgcccccaaggtttact
gatgctccgtttggttcagactgtgagatcactggctttggaaaagagtc
tgaaagtgactatctctatccaaagaacctgaaaatgtccgtcgtaaagc
ttgtttctcatgaacagtgtatgcagccccactactatggctctgaaatt
aattataaaatgctgtgtgctgcggacccagagtggaaaacagattcctg
caagggcgattctggaggaccgcttatctgtaacatcgaaggccgcccaa
ctctgagtgggattgtgagctggggccgaggatgtgcagagaaaaacaag
cccggtgtctacacgagggtctcacacttcctggactggattcaatccca
cattggagaagagaaaggtctggccttctgagatctttttccctctgcca
aaaattatggggacatcatgaagccccttgagcatctgacttctggctaa
taaaggaaatttattttcattgcaatagtgtgttggaattttttgtgtct
ctcactcggaaggacatatgggagggcaaatcatttaaaacatcagaatg
agtatttggtttagagtttggcaacatatgcccatatgctggctgccatg
aacaaaggttggctataaagaggtcatcagtatatgaaacagccccctgc
tgtccattccttattccatagaaaagccttgacttgaggttagatttttt
ttatattttgttttgtgttatttttttctttaacatccctaaaattttcc
ttacatgttttactagccagatttttcctcctctcctgactact.

In the above, the underlined sequence was TRE3G sequence, the wavy-line part was uPA nucleotide sequence, and the italic part was rabbit polyA sequence.

1.5 Preparation of targeting vector

1.5.1 The intermediate vector (Alb-pro) prepared in the preceding step was linearized with Ascl enzyme digestion as a ligation vector.

1.5.2 SLIC method was adopted, ligation 2×HIFI Mix, TRE3G-uPA-rabbit polyA fusion fragment, and linearized intermediate vector (Alb-pro) fragment were added to a sterile tube, which was supplemented with sterile water to 20 μL; the resultant was reacted at 50° C. for 30 min to obtain the targeting vector containing the Rosa26 arm1-Alb enhancer-Alb promoter-Tet-On 3G-HGH polyA-rabbit polyA-uPA-TRE3G-Rosa26 arm2 fragment (named as Alb final vector); after the reaction was ended, Top 10 chemical competence transformation was performed, LB solid agar medium with Spec resistance was coated after Top 10 chemical competence transformation was finished, followed by inverted culturing at 37° C. overnight.

1.5.3 Identification of targeting vector

The monoclonal was picked from a plate into test tubes containing 4 mL LB with Spec resistance and cultured. The bacterial solution was subjected to PCR identification using the primers in Table 10, positive clones (see FIG. 4) obtained from the PCR identification were then subjected to enzyme digestion identification to confirm (expected band sizes after the enzyme digestion identification are shown in Table 11). See FIG. 5 for results. The vectors identified to be correct were targeting vectors, and the correct clones were sequenced using the primers in Table 12 (see Table 12 for primers used for sequencing).

Upon PCR, enzyme digestion identification, and sequencing, the results show that 3#, 7#, and 10# are correct targeting vectors as described in Example 1.

TABLE 10
Primers for Identifying Bacterial Solution
Containing Targeting Vector

| Name of primer | Primer sequence (5′-3′) |
|---|---|
| Alb-seq-F1 | GGTGCAAGCACGTTTCCGACTT |
| Alb-seq-R8 | CCCCTTGAGCATCTGACTTCTG |
| Product size | 715 bp |

TABLE 11
Enzyme Digestion Identification Scheme of Targeting Vector

| Restriction enzyme | Band size |
|---|---|
| PvuII | 4904, 2902, 2514, 290 |
| ApaLI | 6799, 2318, 1019, 874 |

TABLE 12
Primers for Sequencing Rosa26 Alb-tet3G-upA-
enhancer-pro-tetOn3G

| Name of primer | Primer sequence (5′-3′) |
|---|---|
| Alb-seq-F1 | GGTGCAAGCACGTTTCCGACTT |
| Alb-seq-F2 | CCTAGACTACACTACACATTCT |
| Alb-seq-R3 | AGATTGAATTCACCCCTAAGCA |
| Alb-seq-F4 | TGCCACCACTGCCTGGCTACAA |
| Alb-seq-F5 | GAGGTAAGTATGGTTAATGATC |
| Alb-seq-F6 | ATCATATGTGGCCTGGAGAAAC |
| Alb-seq-R7 | GGAGCGGGAGAAATGGATATGA |
| Alb-seq-F8 | TCCAACACACTATTGCAATGAA |
| Alb-seq-F9 | TGACCCAGGTAGACAACGTAGT |
| Alb-seq-F10 | AGGCTCGCCAGCCAGACTTTCA |

The positional relationships of various elements on the targeting vector after successful construction is as shown in FIG. 1.

### Example 3

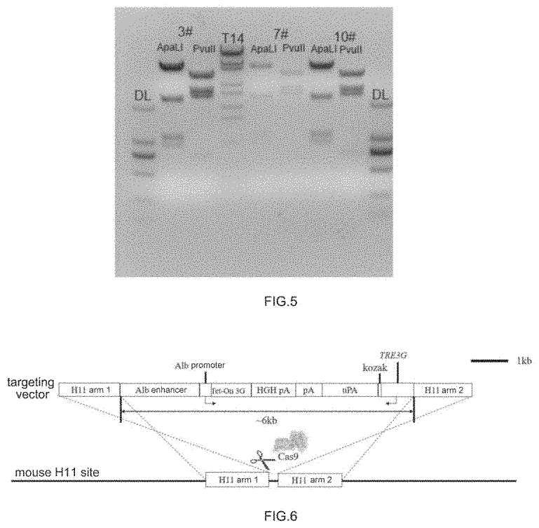

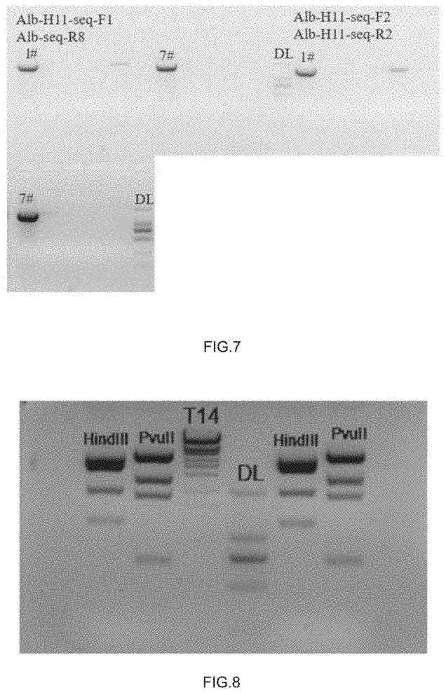

A control targeting vector with mouse genome H11 site as a target insertion site was constructed, and a structural diagram of the elements thereof is shown in FIG. 6. The control targeting vector was different from the targeting vector of Example 1 in homologous arm sequences at the two ends.

A method for constructing the control targeting vector was as follows:

1 preparation of a vector skeleton: amplification was performed using primers in Table 13 with PMD18T-H11-CAG-FLPo (provided by GemPharmatech, Co. Ltd., Jiangsu, see FIG. 19 for a map) as template to obtain a 4965 bp fragment which was recovered for later use, the recovered product was subjected to BglII enzyme digestion, and the resultant was used as a skeleton vector for later use.

TABLE 13
Primers for Amplifying Skeleton Vector

| Name of | | |
|---|---|---|
| primer | Primer sequence (5′-3′) | Note |
| H11-F-BgIII | GAAGATCTGGCGCGCCGTAAGG | BgIII, |
| | GCAGGATGTGTCAAAC | AscI |
| | | enzyme |
| | | digestion |
| | | site |
| H11-R-BgIII | GAAGATCTGGCGCGCCCTCAGC | BgIII, |
| | AGACACCCAGGATAAG | AscI |
| | | enzyme |
| | | digestion |
| | | site |
| Product size | 4965 bp | |
| Template | PMD18T-H11-CAG-FLPo | |

2 The correctly sequenced targeting vectors of Example 1 were subjected to BamHI enzyme digestion, the sizes of the digested fragments were 5851 bp and 5159 bp, respectively, and the 5851 bp fragment was recovered.

3 The above skeleton vector and 5851 bp fragment were ligated using T4 ligase, transformed by Top 10 chemical competence, and coated with Amp-resistant LB solid agar medium after the Top 10 chemical competence transformation was finished, followed by inverted culturing at 37° C. overnight.

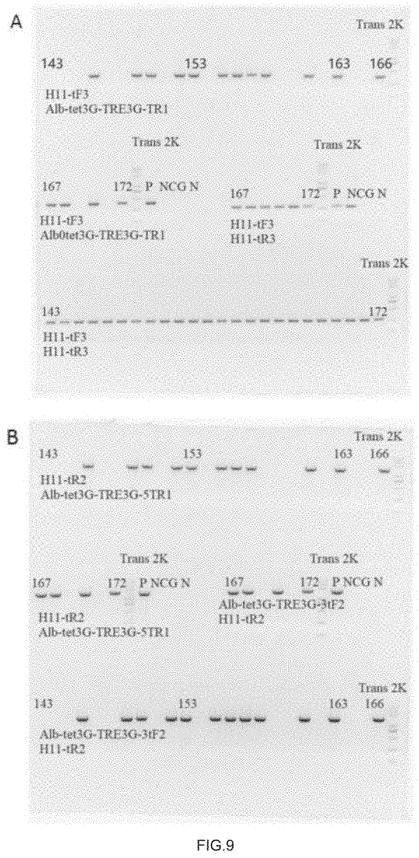

The monoclonal was picked from a plate into test tubes containing 4 mL LB with Amp resistance and cultured. The bacterial solution was subjected to PCR identification using the primers in Table 14. See FIG. 7 for results. PCR positive clones were then subjected to enzyme digestion identification to confirm (expected band sizes after the enzyme digestion are shown in Table 15). See FIG. 8 for results. The clones identified to be correct were control targeting vectors (named as Alb-H11). PCR and enzyme digestion identification results indicate that 1# and 7# are correct Alb-H11 plasmids.

TABLE 14
Primers for PCR Verification for
Alb-H11 Bacterial Solution

| | Primer sequence | Product | |
|---|---|---|---|
| Name of primer | (5′-3′) | size | Note |
| Alb-H11-seq-F1 | TGCAAGGCGATTAAGTTGGGTA | 1652 bp | H1 |
| Alb-seq-R8 | CCCCTTGAGCATCTGACTTCTG | | |
| Alb-H11-seq-F2 | TTGCTGGGATTACAGGCGTGAA | 1474 bp | H2 |
| Alb-H11-seq-R2 | GCGGGCAGTGAGCGCAACGCAA | | |

TABLE 15
Enzyme Digestion Verification Scheme for Alb-H11 Plasmid

| Name of Restriction | |
|---|---|
| enzyme | Band size |
| HindIII | 4013/3391/1895/1156/351 |
| PvuII | 4902/2364/1770/708/690/372 |

In the control targeting vector:

a nucleotide sequence (SEQ ID NO. 12) of one of the chains of the H11 arm1 was as follows:

ctcagcagacacccaggataagtgcactagtgttcctttcctgaccagtg
ggactgctttttccagattcctcttgaagctgaggaggcagtgagagcat
gatggcatctaatgagcttggaagtaccagactgccctgatccacagcca
ggttttgctgaaaagtgaccagtttgtcctcctccagtagagtgggcagc
tgaaggattataatctactgtcaagacttggaggcccctgcagtcaaagt
ccaatagaatattatgaaatggagaatggcttattttaatctctatagtg
gaattaaaatagcatttatggccccagatccataattaatccaatgactg
gtcaaattagcttgaacctgactgaaaaacctgcaatcagtatagtatct
ttcagaatgctttacattcaatttataataccagacttcaagttgtaagt
taacaattttgagagaaactaatgcagcagaggcaagggaaagacttaaa
ttaatgtgaccattaattagttggagtcaggctggattaacattgtgcca
gtaaatttcagaaaaattactatatgacttctctgtaaactttgattttg
tagagtataatattgattccttgatacttgccacaagctactttcagggt
tagtccagcttaaactaatgctatcagaactttatgtgcagaagaaattc
cagaagtcctaaggtaaaattaaaacgtgaaaggaactcatttagactgt
cttagttcatggaagaaataaacacagtaccagccatatcgttcacacac
gtggcatatgtaacttttaataaaccaaaagaaaaagtcccaaatattca
agtgaaaaaaatccaaacagttgacacaagcctaactgatagttacttta
tgtacagctgtatgtataagttcaaaaaaagctaccctatttgtatgtac
aaaagtttatacacaggtctgtacataagggtctatacattttatttttt
cagaacccttaggtgtcacctctagaagacaccaacacttcattcacata
ttttataaaagaaagacttccaggactgacaatcttgtatcccttgtatt
tgaaccatgtaggttcattcgatttaacagccttctgtca.

A nucleotide sequence (SEQ ID NO. 13) of one of the chains of the H11 arm2 was as follows:

tagctcaccttgaaaatggaaacatgtctgacaagagccttgagctgaat
atcatccagagtatttgctagagacagggtcttaagtctcattaaattgc
ttagaagtgtgtttagtcctataaactatgtctcatttgtgtgtccttca
caaagagcgctagtgtcgatcatccattagcctagcctataaaggtgaca
caacctggtcagtccctctgtatgtctactatttccccttctgatttcta
ctatcttctcaagaactggttcttcagcttcctttgggaaatgtcacttt
ttaatgatgtgtgttttgcttatgtatatgtctacatactacccatgtgc
ttggtcactgcagaggccagaagacggtgtcaggtgcactggaactagag
ttaatgacagatgtgagccatagggtgctttgttcacatctttccagtcc
caaatgcgtctaaacttatgtgatacatactagattaccacaaaattgct
ccaaagacacctttctccctctgagatgaatctctggctggccttgctct
aggcaatcctgtgttcaattcaaggactgaatttgattacatcggtaatc
cagtgccccaggcatttctgctttttctgtaaggttcttatcccctggaa
gactgtttacgtattcatttttttttgagactaagtttcaggtagaaaaa
gcttgccttgaacttcactatatagggcttatcttgaactcttgtgggtc
ttccacctttcttcagttagcttctgtacactgccagacatgaaaatcag
atccatttatagatagatggtcatgagccaccatgtgggtgtctaaaatt
gatctcaggacctctgaaagaccagctagttctcttaactgctgagccat
ctctctagcgtgtctatacacatttaatatccccttgttccctttctgct
tcatcttgctgatcatgattagtgtttgcctttgttacctgttccatcag
cttcagcctgaagagtaagtagttctctattggcagtttgacacatcctg
cccttac.

### Example 4

The constructed vector (targeting vector or control targeting vector) and the Cas9 system formed an injection system. See the following table:

| | | Final |
|---|---|---|
| Component | Volume (μl) | concentration |
| Targeting vector | 2.6 | 100 ng/μl |
| Cas9-Protein | 1 | 2.5 μg/μl |
| sgRNA | 1 | 1000 ng/μl |
| Injection Buffer | 15.4 | |

The target sequence of sgRNA used for the targeting vector was:

| (SEQ ID NO. 9) | |
|---|---|
| | AGTCTTCTGGGCAGGCTTAA. |

The target sequence of sgRNA used for the control targeting vector was:

| (SEQ ID NO. 14) | |
|---|---|
| | CTGAGCCAACAGTGGTAGTA. |

The injection system was injected into 0.5-day (12 h from the time when sperm was added to an egg culture dish) fertilized eggs (1-2 PL/embryo) of NCG wild-type mice by microinjection, and embryos were transplanted (embryos were implanted by means of oviduct transplantation) into 0.5-day (12 h from the time when the female mice successfully mated with ligated male mice (vaginal plug was successfully detected)) pseudo-pregnant female mice. After the birth of mice, targeted mice were screened out through gene identification, and positive mice were named as: H11-alb-Tet On3G-uPA (constructed using the control targeting vector of Example 3) and Rosa26-alb-Tet-On3G-uPA (constructed using the targeting vector of Example 1). The positive F0 mice were backcrossed with background mice (NCG mice) to obtain F1, and tails of F1 generation mice were subjected to gene identification. The positive F1 mice were bred and established for verification experiments.

TABLE 16
List of Primers for Identification of H11-alb-Tet
On3G-uPA Mouse

| Serial | Name of | | |
|---|---|---|---|
| No. | primer | Primer sequence | Band size |
| 1 | H11-tF3 | GGGCAGTCTGGTACTTCCA | KI: 398 bp |
| | | AGCT | |
| | Alb-tet3G- | GTGTGGGTTGCCACCTCTT | Wt: none |
| | TRE3G-TR1 | TAG | |
| 2 | H11-tF3 | GGGCAGTCTGGTACTTCCA | KI: none |
| | | AGCT | |
| | H11-tR3 | ATATCCCCTTGTTCCCTTT | Wt: 285 bp |
| | | CTGC | |
| 3 | H11-tF2 | ATGCCCACCAAAGTCATCA | KI: 1594 bp |
| | | GTGTAG | |
| | Alb-tet3G- | GTGTGGGTTGCCACCTCTT | Wt: none |
| | TRE3G-5TR1 | TAG | |
| 4 | Alb-tet3G- | GCGATCTGACGGTTCACTA | KI: 1595 bp |
| | TRE3G-3tF2 | AACGAG | |
| | H11-tR2 | TCACAGAAACCATATGGCG | Wt: none |
| | | CTCC | |

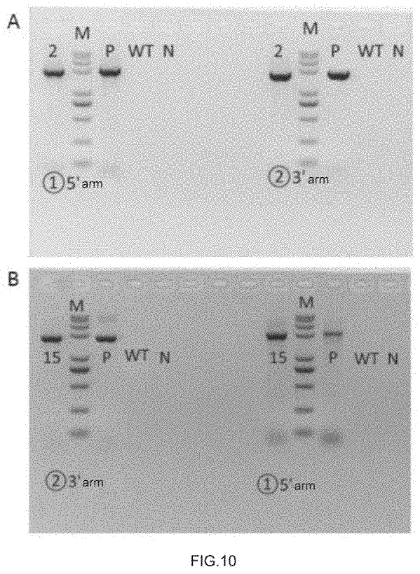

Identification results in FIG. 9 show that 146#, 149#, 150#, 152#, 153#, 155#, 156#, 157#, 158#, 161#, 163#, 166#, 167#, 168#, 170#, 172# are F1 generation positive H11-alb-Tet On3G-uPA heterozygous mice (ki/wt), and the remaining are wild type.

TABLE 17
List of Primers for Identification of Rosa26-
alb-Tet-On3G-uPA F1 Generation Mouse

| Serial | Name of | | Product |
|---|---|---|---|
| No. | primer | Primer sequence | size |
| [symbol] 5′ | upA-tF1 | TGGAGCTCCTGATGAATCAA | WT: 0 bp |
| arm | | ACT | |
| | ypA-tR1 | ATAGCACCAGGGTCGCTTCT | Targeted: |
| | | | 1979 bp |
| [symbol] 3′ | Tet on3G- | ACAGGCATCATACCCACTCC | WT: 0 bp |
| arm | F1 | | |
| | Tet on3G- | GCGTCAGCAGGCAGCATA | Targeted: |
| | R2 | | 1749 bp |

Identification results in FIG. 10 show that mouse No. 15 is F1 generation positive Rosa26-alb-Tet-On3G-uPA heterozygous mouse.

### Example 5

Detection of Liver Injury Law of Mice

1 Detection of Liver Injury Law of H11-Alb-Tet On3G-uPA Mice

Background mice (WT), H11-alb-Tet On3G-uPA heterozygous mice and homozygous mice of 6-8 weeks of age were intragastrically administered or administered by drinking water with 2.0 mg/mL Dox, and orbital blood was sampled at different time points to detect ALT activity levels in serum.

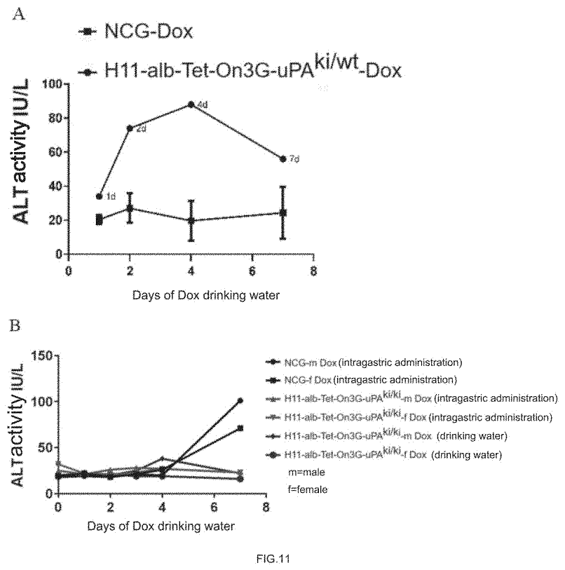

Experimental results in FIG. 11 show that: no matter H11-alb-Tet On3G-uPA heterozygous mice or homozygous mice, after administration of Dox, the ALT of the mice is raised, but the serious liver injury level is still not reached.

2 Detection of Liver Injury Law of Rosa26-Alb-Tet-On3G-uPA Mice

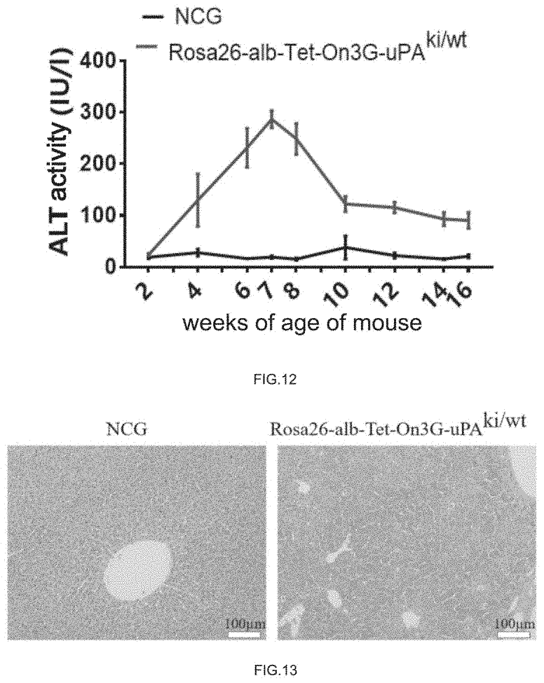

NCG background mice and Rosa26-alb-Tet-On3G-uPA heterozygote mice of 2 weeks of age were subjected to orbital blood sampling at 2, 4, 6, 8, 10, 12, 14, and 16 weeks of age, respectively, and ALT activity in serum was detected. Surprisingly, the Rosa26-alb-Tet-On3G-uPA heterozygous mice had elevated ALT activity at 3-4 weeks of age, and the ALT activity increased with increasing weeks of age, and declined sharply at 8-10 weeks of age (see FIG. 12).

The livers of Rosa26-alb-Tet-On3G-uPA mice of 4 weeks of age were taken, and fixed by paraformaldehyde, followed by paraffin embedding, sectioning, and HE staining analysis. Compared with NCG background mice, Rosa26-alb-Tet-On3G-uPA mice at 4 weeks of age exhibited severe liver injury, hepatocyte ballooning degeneration, localized necrosis, and cytosolic lysis (see FIG. 13).

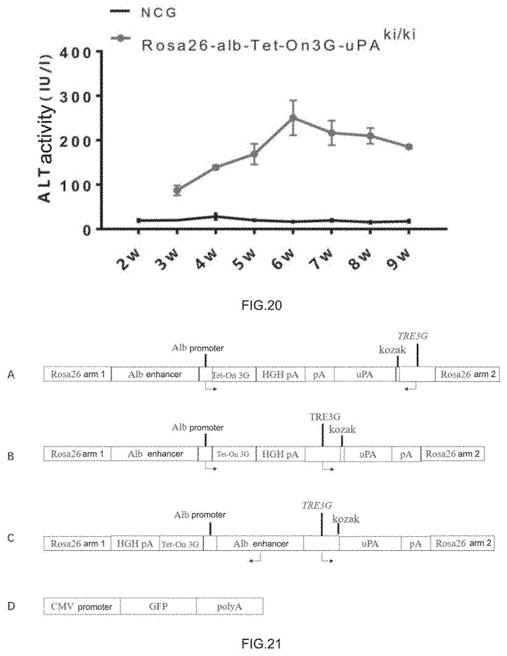

From the above results, it is obtained that the Rosa26-alb-Tet-On3G-uPA heterozygous mice can have spontaneous liver injury, and the degree of liver injury can meet the implantation requirements of exogenous hepatocytes. Rosa26-alb-Tet-On3G-uPA homozygous mice also might have spontaneous liver injury (see FIG. 20).

3 Rosa26-Alb-Tet-On3G-uPA Mice of Different Weeks of Age all Responded to Dox.

3-4-week-old and 6-8-week-old Rosa26-alb-Tet-On3G-uPA mice were taken, and administered with drinking water containing 1-2 mg/mL Dox. Blood was sampled on days 0, 3, 5, and 7 (3-4 weeks old) and days 0, 2, 4, 6, and 8 (6-8 weeks old), to detect the ALT activity in serum. The mice were euthanized on day 7 or 8, and the livers of the mice were taken, fixed, paraffin-embedded, sectioned, and HE-stained, to analyze the situation of hepatocyte injury.

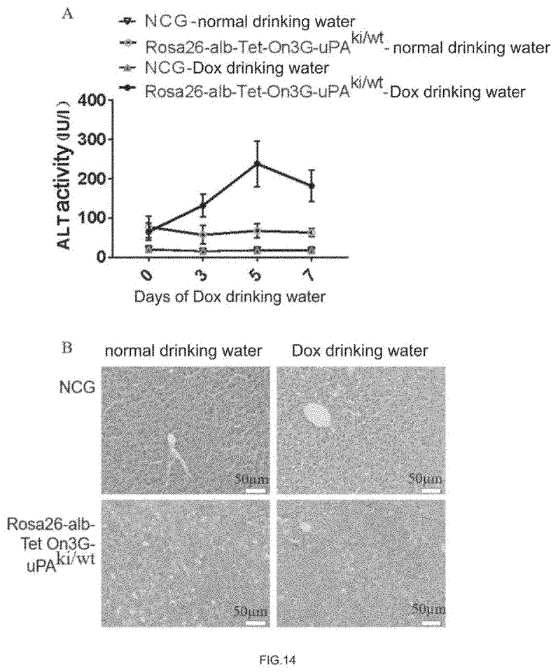

Results are shown in FIG. 14 and FIG. 15: Rosa26-alb-Tet-On3G-uPA mice of both 3-4 weeks of age and 6-8 weeks of age could respond to Dox. Compared with Rosa26-alb-Tet-On3G-uPA mice with common drinking water, the mice with Dox drinking water had obviously elevated ALT activity and aggravated liver injury.

Combining the above results, it can be seen that: compared with the H11-alb-Tet On3G-uPA mice, Rosa26-alb-Tet-On3G-uPA mice had more sensitive responses to Dox, and might have spontaneous injury. Therefore, the Rosa26-alb-Tet-On3G-uPA mice can be used for liver reconstruction after liver injury.

### Example 6

Hepatocyte Transplantation Test

1 Green Fluorescent Hepatocyte Isolation

Male B6-G/R (provided by GemPharmatech, Co. Ltd., Jiangsu, strain No.

T006163) mice of 5-6 weeks of age were chosen. After the mice were anaesthetized, abdomens were washed with alcohol. The abdominal cavities were cut open to expose inferior vena cava and portal vein, and a retention needle was inserted from the inferior vena cava. After successful pre-perfusion, portal vein was cut open. Pre-heated Preperfusion buffer-P1 (HBSS formulated EDTA solution with a final concentration of 5 mM) and Enzyme buffer-P2 (HBSS formulated CaCl₂ solution (pH7.2) with a final concentration of 5 mM, with Collagenase added prior to use) were perfused in sequence. After the perfusion was finished, the whole liver was removed, washed by PBS, and then put into a P2 solution, a liver capsule was scraped, to let the hepatocytes enter P2, and the cell suspension was filtered. To the filtrate DMEM with 10% FBS was added, followed by centrifugation at 400 rpm for 3 min at 4° C., and the supernatant was removed.

To the above precipitation, a percoll mixture (10 mL DMEM+1 mL 10×PBS+9 mL percoll) was added to resuspend the cells, followed by centrifugation at 1100 rpm for 3 min at 4° C., and the supernatant was removed.

The cells were washed with precooled DMEM, the cell viability was detected, and the cells were diluted to a proper concentration according to a counting result for later use.

2 Mouse Spleen In Situ Hepatocyte Transplantation

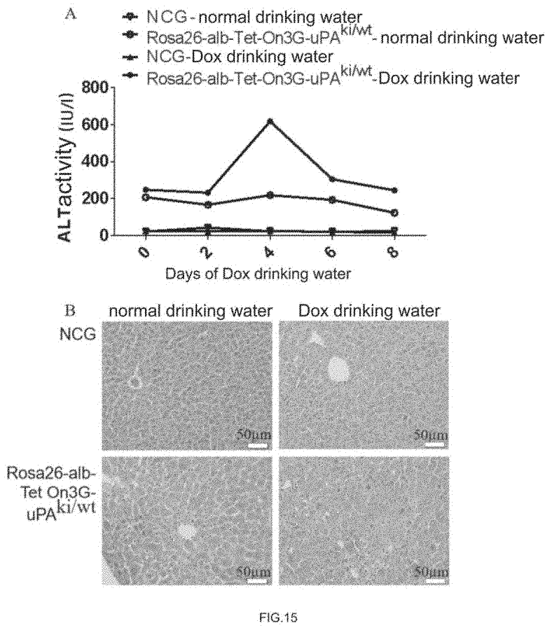

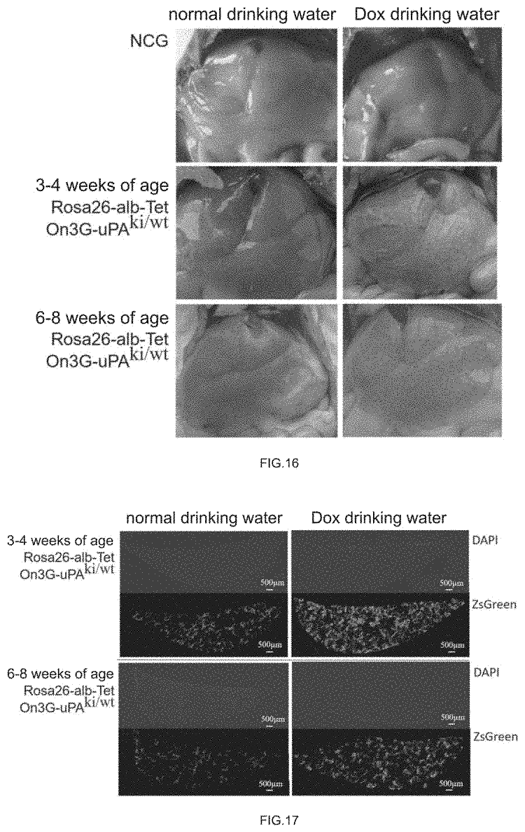

The Rosa26-alb-Tet-On3G-uPA mice and the NCG mice were randomly divided into the following groups (see Table 18). Each mouse was injected with fresh green fluorescent hepatocytes by intrasplenical transplantation. After completing the transplantation procedure, the mice were incubated on a hot stage at 37° C. After natural awakening, the mice were injected with antibiotics, and the mice were returned to a feeding cage, and administered with normal drinking water or Dox drinking water according to the requirements in the following table. 10 weeks after hepatocyte transplantation of the mice was completed, all the mice were subjected to end-point sampling. The livers were fixed and embedded, and the distribution of the mouse green fluorescent cells was observed through frozen sections. See FIG. 16 and FIG. 17 for results.

TABLE 18
Mouse Hepatocyte Transplantation and Condition Treatment Grouping

| | | | | Drinking water condition |
|---|---|---|---|---|
| | | | Number | in 1 week after |
| Group | Mouse genotype | Weeks of age | of mice | transplantation experiment |
| G1 | NCG | 3-4 weeks of age | 8 | Normal drinking water |
| G2 | Rosa26-alb-Tet-On3G-uPA | 3-4 weeks of age | 8 | Normal drinking water |
| G3 | NCG | 3-4 weeks of age | 8 | DOX drinking water |
| G4 | Rosa26-alb-Tet-On3G-uPA | 3-4 weeks of age | 8 | DOX drinking water |
| G5 | NCG | 6-8 weeks of age | 8 | Normal drinking water |
| G6 | Rosa26-alb-Tet-On3G-uPA | 6-8 weeks of age | 8 | Normal drinking water |
| G7 | NCG | 6-8 weeks of age | 8 | DOX drinking water |
| G8 | Rosa26-alb-Tet-On3G-uPA | 6-8 weeks of age | 8 | DOX drinking water |

Results in FIG. 16 and FIG. 17 show that:

(1) After the 3-4-week-old and 6-8-week-old Rosa26-alb-Tet-On3G-uPA mice were subjected to hepatocyte transplantation, the mice were administered with normal drinking water for one week, the exogenous hepatocytes could be uniformly distributed on the liver surface of the mice, and the reconstruction rate might reach 50%.

(2) After 3-4-week-old and 6-8-week-old Rosa26-alb-Tet-On3G-uPA mice were subjected to hepatocyte implantation, the mice were administered with Dox drinking water for one week, the green fluorescent hepatocytes might be uniformly distributed on the liver surface, and the reconstruction rate might reach 90%; compared with the normal drinking water group, giving the mice Dox drinking water might obviously improve the colonization of exogenous hepatocytes in the mouse bodies.

### Example 7

Detecting the survival rate of Rosa26-alb-Tet-On3G-uPA mice

Offspring Rosa26-alb-Tet-On3G-uPA heterozygote mice were obtained by mating the Rosa26-alb-Tet-On3G-uPA heterozygote mice with NCG background mice for experiments. 1335 Rosa26-alb-Tet-On3G-uPA heterozygote mice were obtained, 1299 mice survived at the perinatal period, with a survival rate up to 97%, the survival rate of the Rosa26-alb-Tet-On3G-uPA homozygous mice was similar to that of the Rosa26-alb-Tet-On3G-uPA heterozygote mice, and compared with the survival rate (70%-75%) of uPA-SCID mice in the perinatal period, the method of the examples of the present disclosure greatly improves the survival rate of mice.

To sum up the results, it can be seen that:

an example of the present disclosure provides a vector (Example 1) carrying Tet on system with Rosa26-site homologous arm and regulating specific expression of uPA in liver. The vector can be integrated into the Rosa26 site at a fixed site, and the mouse model Rosa26-alb-Tet-On3G-uPA constructed using the vector combined by such elements avoids interference effect of adjacent sequences on the genome, and will not destroy any endogenous gene, thus ensuring the normal growth and development, and normal function of mice.

Surprisingly, the mouse model Rosa26-alb-Tet-On3G-uPA constructed using the vector could have spontaneous liver injury (liver injury appeared at 2-3 weeks of age, and the ALT value might be up to 300 IU/L at 8 weeks of age), and the level of spontaneous liver injury completely satisfies the implantation requirements of the exogenous hepatocytes, that is, colonization of the exogenous hepatocytes can be achieved without Dox induction, so that the transplantation procedure is more simplified. This spontaneous liver injury phenotype breaks the conventional knowledge of characteristics for the Tet on system to regulate protein expression.

In addition, on the basis of spontaneous liver injury, Rosa26-alb-Tet-On3G-uPA can respond to Dox at different growth stages, that is, with Dox induction, the liver injury level of mice might be aggravated, and the colonization rate of exogenous hepatocytes can be improved.

In combination with the characteristics that the Rosa26-alb-Tet-On3G-uPA mice could have spontaneous liver injury and the liver injury might be aggravated by Dox induction, mice of different weeks of age could be selected at will for liver reconstruction using the characteristic of spontaneous liver injury; or the colonization rate of exogenous hepatocytes can be improved by Dox induction. Using Rosa26-alb-Tet-On3G-uPA mice for liver reconstruction, the transplantation window is no longer limited by the weeks of age, and the condition of transplantation and reconstitution is more flexible.

In addition, it also should be noted that the mortality of the heterozygote offspring of the Rosa26-alb-Tet-On3G-uPA mice provided by the example of the present disclosure is much lower than that of existing spontaneously liver-injured mouse models, which lays a foundation for large-scale use of liver-injured mouse models.

### Example 8

The present example provides the influence of different ligation relationships between the first expression cassette and the second expression cassette on the hepatoma cell line HepG2, including the following steps:

preparing expression vectors with different ligation relationships (tail-to-tail (A), tail-to-head (B), and head-to-head (C)) between the first expression cassette and the second expression cassette with the Rosa26 arm1-Alb enhancer-Alb promoter-Tet-On 3G-HGH polyA-rabbit polyA-uPA-RE3G-Rosa26 arm2 in Example 1 as template, respective target sequences, and a 5′ end homologous arm sequence (Rosa26 arm1) and a 3′ end homologous arm sequence (Rosa26 arm2) for mediating insertion of the target sequence into a target site (Rosa26) in a mouse genome (see FIG. 21 for positional relationship of various elements).

(1) Resuscitation of HepG2 cells and amplification.

(2) Electrotransformation of HepG2 cells, including adding DPBS to a petri dish to wash the cells and removing the supernatant, digesting and obtaining HepsG2 cells, counting the cells and then equally dividing the cells into 20 equal parts (1.5×10⁶/part), adding an electrotransformation solution and plasmids with different ligation relationships to the cells, respectively, mixing the same uniformly, and transferring the resultant to an electrode cup for electrotransformation, after the end of the electrotransformation, quickly sucking out the cell suspension, and placing the cell suspension in the petri dish for culturing.

(3) 12 h after the electrotransformation, removing the medium and suspended dead cells, and adding a normal medium to continue the culturing for 12 h.

(4) 24 h after the electrotransformation, changing to a normal medium or medium adding with Dox according to the processing manner in Table 19, to continue the culturing.

TABLE 19
Influence of Vectors Having Two Expression Cassettes in
Different Ligation Relationships on HepG2 Hepatoma Cells and
Condition Treatment Grouping

| | | | Observation Time |
|---|---|---|---|
| | Name of Vector | | Point after |
| Group | Transferred | Culture Condition | Electrotransformation |
| G1 | Tail-to-tail plasmid | normal medium | 24h; 48h; |
| G2 | Tail-to-tail plasmid | normal | 24h; 48h; |
| | | medium + Dox | |
| G3 | Tail-to-head | normal medium | 24h; 48h; |
| | plasmid | | |
| G4 | Tail-to-head | normal | 24h; 48h; |
| | plasmid | medium + Dox | |
| G5 | head-to-head | normal medium | 24h; 48h; |
| | plasmid | | |
| G6 | head-to-head | normal | 24h; 48h; |
| | plasmid | medium + Dox | |
| G7 | GFP plasmid | normal medium | 24h; 48h; |
| G8 | GFP plasmid | normal | 24h; 48h; |
| | | medium + Dox | |

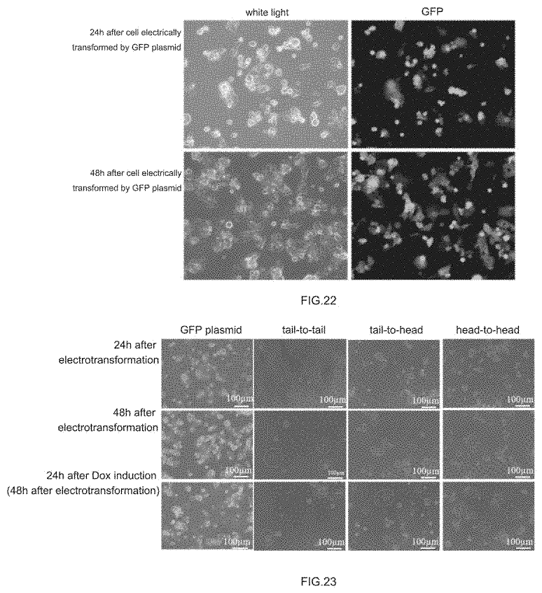

Microscopic imaging observation was performed 24 h and 48 h after cell electrotransformation was completed, respectively, and results are shown in FIG. 22 and FIG. 23.

(1) It can be seen from FIG. 22 that 24 h and 48 h after the GFP plasmid was electrically transformed to HepsG2 cells, the HepsG2 cells could all express green fluorescence, which indicates that the plasmid can be integrated into the cell genome by electrotransformation and expressed, and expression vectors with different ligation relationships can enter the HepsG2 cells through electrotransformation and expressed.

(2) FIG. 23 shows that 24 h after the cell electrotransformation was completed, compared with plasmids with other ligation relationships, the plasmid having the first expression cassette and the second expression cassette in a tail-to-tail ligation relationship has the maximum damage to the HepsG2 cells. It can be seen that the number of adherent cells is significantly less than that of cells electrically transformed by plasmids with other ligation relationships, and the cells were in an apoptotic state (cells in the red circle are abnormal in morphology, and have increased antennae), indicating that the plasmid having the first expression cassette and the second expression cassette in the tail-to-tail ligation relationship may express uPA without Dox induction, thus causing cell injury and apoptosis, then leakage expression of uPA may exist in this ligation manner.

(3) FIG. 23 also shows that after the HepsG2 cells were cultured with the medium containing Dox for 24 h, the HepsG2 cells electrically transformed by plasmids with different ligation relationships all had abnormal morphology, and increased antennae, and were in apoptosis state, indicating that the plasmids with different ligation relationships may all respond to Dox induction.

### Example 9

Influence of different ligation relationships between the first expression cassette and the second expression cassette on mouse liver injury

The hydrodynamic-based method can efficiently introduce an exogenous gene into the liver, has been widely applied to gene function research, and is selected for treating diseases of liver and other organs. Rapid injection of large amounts of DNA at tail vein under the high pressure results in extremely high expression levels of endogenous genes in vivo, particularly in hepatocytes. However, at the same time, high hydraulic pressure also will cause serious injury to the liver (increase of ALT).

The present example provides the influence of different ligation relationships between the first expression cassette and the second expression cassette on the mouse liver injury, including the following steps.

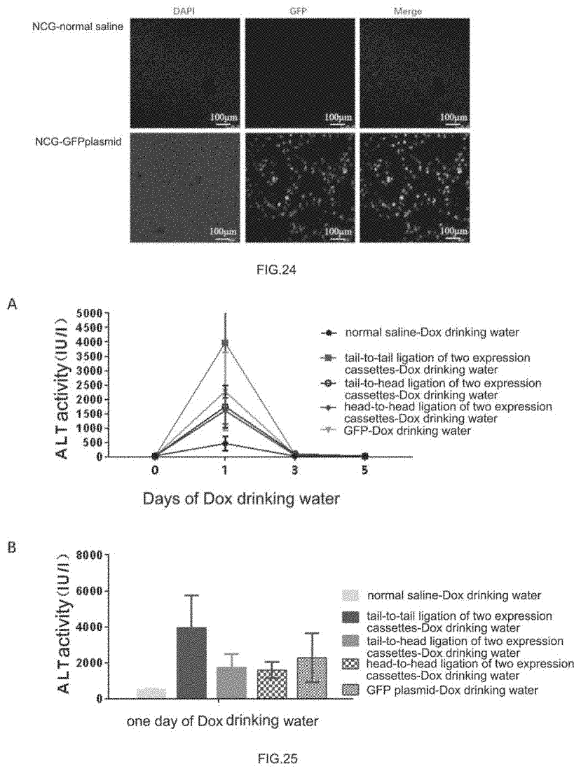

The expression vectors with different ligation relationships prepared in the above were injected into mouse bodies in a manner of high-pressure tail-vein rapid injection, meanwhile GFP (Pmax-GFP) plasmids that can express green fluorescence and normal saline were injected as control. After the mice were injected with the plasmid, Dox drinking water (2 mg/L) was administered, orbital blood was sampled on days 1, 3, 5, and 7, respectively, and ALT activity in serum was detected. A specific procedure is as shown in Table 20. On Day 1 after the injection was completed, the livers of the mice in the group injected with GFP plasmid and normal saline were taken, fixed, and embedded, and the distribution of mouse green fluorescent cells was observed through frozen sections. See FIG. 24 for results.

TABLE 20
Condition Treatment Grouping of Experiment for Influence of
Vectors with Different Ligation Relationships on Mouse Liver Injury

| | Ligation | Number | Amount of | Blood |
|---|---|---|---|---|
| | Relentionship between | of | plasmids | sampling |
| Group | Two Expression Cassettes | animals | injected | time |
| 1 | tail-to-tail ligation of two | 6 | 20 μg | D-1/1/3/5/7 |
| | expression cassettes | | | |
| 2 | tail-to-head ligation of two | 6 | 20 μg | D-1/1/3/5/7 |
| | expression cassettes | | | |
| 3 | head-to-head ligation of two | 6 | 20 μg | D-1/1/3/5/7 |
| | expression cassettes | | | |
| 4 | GFP plasmid | 6 | 20 μg | D-1/1/3/5/7 |
| 5 | none | 6 | normal | D-1/1/3/5/7 |
| | | | saline | |

Results in FIG. 24 and FIG. 25 show that:

(1) After GFP plasmid was injected, the mouse liver can express green fluorescence, indicating that plasmid DNA injected through tail vein can be expressed in hepatocytes.

(2) After the plasmids with different ligation relationships and GFP plasmid were injected and when Dox drinking water was administered to the mice, only the ALT of the mice injected with the plasmids with tail-to-tail ligation relationship was obviously increased. Therefore, the vector with the first expression cassette and the second expression cassette in a tail-to-tail ligation relationship can cause the mice to generate liver injury under Dox induction, which is superior to other ligation relationships.

In conclusion, the two expression cassettes in the tail-to-tail ligation relationship can clearly respond to Dox, which is superior to other ligation relationships.

The above-mentioned are merely for preferred examples of the present disclosure and not intended to limit the present disclosure. For one skilled in the art, various modifications and variations may be made to the present disclosure. Any modifications, equivalent substitutions, improvements and the like made within the spirit and principle of the present disclosure are intended to be included within the scope of protection of the present disclosure.

### INDUSTRIAL APPLICABILITY

The present disclosure provides a targeting vector, a nucleic acid composition, and a method for constructing a liver-injured mouse model. The liver-injured mouse model constructed using the targeting vector provided in the present disclosure can generate liver injury without induction of an inducer, it can spontaneously generate the liver injury, and the degree of liver injury can be enhanced with use of the inducer. In addition, the degree of spontaneous liver injury of the liver-injured mouse model not only can satisfy the transplantation requirements of exogenous hepatocytes, but also the reconstruction rate is high after hepatocyte transplantation. Besides, the liver-injured mouse model can breed offspring liver-injured mice by crossbreeding, and the mortality of offspring mice is low, facilitating large-scale breeding. The present disclosure provides reliable liver-injured mouse models for studies of liver diseases.

1. A targeting vector for constructing a liver-injured mouse model, wherein the targeting vector contains a target sequence, and a 5′ end homologous arm sequence and a 3′ end homologous arm sequence for mediating insertion of the target sequence into a target site in a mouse genome, wherein the target sequence comprises a first expression cassette and a second expression cassette located downstream of the first expression cassette,
wherein the first expression cassette has following elements connected in series in sequence: a liver-specific promoter, a tetracycline transcription activation regulating factor and a first polyA; and the second expression cassette has following elements connected in series in sequence: a second polyA, a mouse prourokinase activator encoding gene and a tetracycline-inducible promoter,
wherein the liver-specific promoter drives expression of the tetracycline transcription activation regulating factor in a direction from upstream to downstream, and the tetracycline-inducible promoter drives expression of the mouse prourokinase activator encoding gene in a direction from downstream to upstream, wherein the target site is Rosa26 site.

2. The targeting vector according to claim 1, wherein the first expression cassette further has an enhancer sequence, wherein the enhancer sequence is located upstream of the liver-specific promoter.

3. The targeting vector according to claim 2, wherein the liver-specific promoter is any one selected from the group consisting of albumin promoter, apolipoprotein E promoter, phosphoenolpyruvate carboxykinase promoter, α-1-antitrypsin promoter, thyroxin binding globulin promoter, α-fetoprotein promoter, alcohol dehydrogenase promoter, IGF-II promoter, factor VIII promoter, HBV core protein promoter, HBV pre-s2 protein promoter, thyroxine-binding globulin promoter, HCR-ApOCII hybrid promoter, HCR-hAAT hybrid promoter, AAT promoter combined with enhancer element of mouse albumin gene, low-density lipoprotein promoter, pyruvate kinase promoter, lecithin-cholesterol acyltransferase promoter, apolipoprotein H promoter, transferrin promoter, transthyretin promoter, α-fibrinogen and β-fibrinogen promoter, α-I-antichymotrypsin promoter, α-2-HS glycoprotein promoter, haptoglobin promoter, ceruloplasmin promoter, plasminogen promoter, complement protein promoter, promoter of complement C3 activator, hemopexin promoter and α-I-acid glycoprotein promoter.

4. The targeting vector according to claim 2, wherein the liver-specific promoter is an albumin promoter.

5. The targeting vector according to claim 2, wherein the enhancer sequence is an albumin enhancer.

6. The targeting vector according to claim 2, wherein the tetracycline transcription activation regulating factor is any one selected from the group consisting of tTA, rtTA and Tet-On 3G.

7. The targeting vector according to claim 6, wherein the tetracycline transcription activation regulating factor is Tet-On 3G.

8. The targeting vector according to claim 2, wherein the first polyA is selected from the group consisting of HGH polyA, SV40 polyA, BGH polyA, rbGlob polyA, SV40 late polyA and rbGlob polyA.

9. The targeting vector according to claim 1, wherein in the second expression cassette, a Kozak sequence is further inserted between the mouse prourokinase activator encoding gene and the tetracycline-inducible promoter.

10. The targeting vector according to claim 9, wherein the tetracycline-inducible promoter is any one selected from the group consisting of TRE3G and TetO6.

11. (canceled)

12. The targeting vector according to claim 9, wherein an amino acid sequence of a mouse prourokinase activator encoded by the mouse prourokinase activator encoding gene is represented by SEQ ID NO. 7.

13. The targeting vector according to claim 12, wherein a nucleotide sequence of the mouse prourokinase activator encoding gene is represented by sites 1-1302 in SEQ ID NO. 6 or a complementary sequence thereof.

14. The targeting vector according to claim 9, wherein the second polyA is selected from the group consisting of rabbit polyA, SV40 polyA, hGH polyA, BGH polyA, rbGlob polyA, SV40 late polyA and rbGlob polyA.

15. (canceled)

16. The targeting vector according to claim 1, wherein the 5′ end homologous arm sequence is represented by SEQ ID NO. 4 or a complementary sequence thereof; and the 3′ end homologous arm sequence is represented by SEQ ID NO. 5 or a complementary sequence thereof.

17. A nucleic acid composition for constructing a liver-injured mouse model, comprising the targeting vector according to claim 1 and a CRISPR/Cas9 composition for double-strand breakage of a mouse genome sequence at the target site.

18. The nucleic acid composition according to claim 17, wherein the CRISPR/Cas9 composition comprises: Cas9 protein and sgRNA.

19. The nucleic acid composition according to claim 18, wherein a target sequence of the sgRNA is represented by SEQ ID NO. 9.

20. (canceled)

21. (canceled)

22. A method for constructing a liver-injured mouse model, wherein the target sequence is inserted into the target site on a genome of a target mouse using the targeting vector according to claim 1.

23. The method according to claim 22, wherein the target mouse is a mouse having immunodeficiency.

24. The method according to claim 23, wherein the method comprises: injecting the nucleic acid composition into fertilized eggs from mice having immunodeficiency, then transplanting the fertilized eggs into bodies of pseudo-pregnant female mice, and screening out, from offspring of the pseudo-pregnant female mice, positive mice with a genome inserted with the target sequence, to obtain the liver-injured mouse model.

25. (canceled)

26. (canceled)

27. (canceled)

28. (canceled)

29. (canceled)

30. (canceled)
